(19) United States
(12) Patent Application Publication
(10) Pub. No.: US 2004/0102325 A1
(43) Pub. Date: May 27, 2004

(54) Phenylpropynyloxypyridine herbicides

(76) Inventors: J?uuml;rgen Schaetzer, (US); Peter Renold, Basel (CH); Gerald Wayne Craig, Basel (CH); Martin Eberle, Bottmingen (CH); Roger Graham Hall, Basel (CH)

Correspondence Address: SYNGENTA CROP PROTECTION , INC., PATENT AND TRADEMARK DEPARTMENT, 410 SWING ROAD, GREENSBORO, NC, 27409, US

(21) Appl. No.: 10/381,572

(22) PCT Filed: Oct. 1, 2001
(86) PCT No.: PCT/EP01/11353

(30) Foreign Application Priority Data
Oct. 3, 2000 (CH) 1937/00

(51) Int. Cl. A01N 43/40; C07D 213/72; C07D 213/62; C07D 213/78

(52) U.S. Cl. 504/251; 504/254; 546/268.1; 546/288; 546/298; 546/297

(57) ABSTRACT

Compounds of formula (I) wherein the substituents R₁, R₂, R₃ and R₄, and the suffixes n and m are as defined in claim 1, and agrochemically acceptable salts and all stereoisomers and tautomers of such compounds are suitable for use as herbicides.

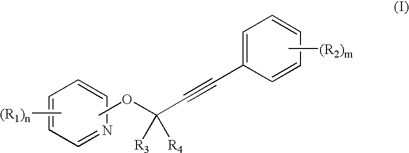

The present invention relates to novel, herbicidally active phenylalkynes, to a process for their preparation, to compositions comprising such compounds, and to the use thereof in controlling weeds, especially in crops of useful plants, or in inhibiting plant growth.

Phenylalkynes having herbicidal action are described, for example, in JP-A-11 147 866.

Novel phenylalkynes having herbicidal and growth-inhibiting properties have now been found.

The present invention accordingly relates to compounds of formula I

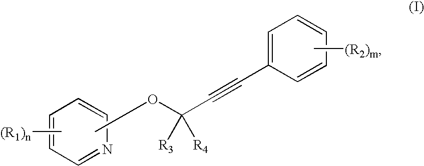

wherein

n is 0, 1, 2, 3 or 4;

each R₁ independently of any other(s) is halogen, —CN, —SCN, —SF₅, —NO₂, —NR₅R₆, —CO₂R₇, —CONR₈R₉, —C(R₁₀)═NOR₁₁, —COR₁₂, —OR₁₃, —SR₁₄, —SOR₁₅, —SO₂R₁₆, —OSO₂R₁₇, C₁C₈alkyl, C₂-C₈alkenyl, C₂-C₈alkynyl or C₃-C₆cycloalkyl; or C₁-C₈alkyl, C₂-C₈alkenyl or C₂C₈alkynyl substituted by one or more halogen, —CN, —NO₂, —NR₁₈R₁₉, —CO₂R₂₀, —CONR₂₁R₂₂, —COR₂₃, —C(R₂₄)═NOR₂₅, —C(S)NR₂₆R₂₇, —C(C₁-C₄alkylthio)=NR₂₈, —OR₂₉, —SR₃₀, —SOR₃₁, —SO₂R₃₂ or C₃-C₆cycloalkyl substituents; or

each R₁ is C₃-C₆cycloalkyl substituted by one or more halogen, —CN, —NO₂, —NR₁₈R₁₉, —CO₂R₂₀, —CONR₂₁R₂₂, —COR₂₃, —C(R₂₄)═NOR₂₅, —C(S)NR₂₆R₂₇, —C(C₁-C₄alkylthio=NR₂₈, —SR₃₀, —SOR₃₁, —SO₂R₃₂ or C₃-C₆cycloalkyl substituents; or

each R₁ independently of any other(s) is phenyl, which may itself be substituted by one or more halogen, C₁-C₄alkyl, C₁-C₄haloalkyl, C₁-C₄alkoxy, —CN, —NO₂, C₁-C₄alkylthio, C₁-C₄-alkylsulfinyl or C₁-C₄alkylsulfonyl substituents; or

two adjacent R₁ together form a C₁-C₇alkylene bridge, which may be interrupted by 1 or 2 non-adjacent oxygen atoms and may be substituted by C₁-C₆alkyl, the total number of ring atoms being at least 5 and a maximum of 9; or two adjacent R₁ together form a C₂-C₇-alkenylene bridge, which may be interrupted by 1 or 2 non-adjacent oxygen atoms and may be substituted by C₁-C₆alkyl, the total number of ring atoms being at least 5 and a maximum of 9;

R₃ and R₄ are each independently of the other hydrogen, halogen, —CN, C₁-C₄alkyl or C₁-C₄-alkoxy; or

R₃ and R₄ together denote C₂-C₅alkylene;

R₅ is hydrogen or C₁-C₈alkyl;

R₆ is hydrogen, C₁-C₈alkyl, C₃-C₈alkenyl, C₃-C₈alkynyl, phenyl or benzyl; it being possible for phenyl and benzyl themselves to be substituted by one or more halogen, C₁-C₄alkyl, C₁-C₄-haloalkyl, C₁-C₄alkoxy, —CN, —NO₂, C₁-C₄alkylthio, C₁-C₄alkylsulfinyl or C₁-C₄alkylsulfonyl substituents; or

R₅ and R₆ together denote a C₂-C₅alkylene chain, which may be interrupted by an oxygen or sulfur atom;

R₇ is hydrogen, C₁-C₈alkyl, C₃-C₈alkenyl or C₃-C₈alkynyl, or C₁-C₈alkyl, C₃-C₈alkenyl or C₃-C₈alkynyl substituted by one or more halogen, C₁-C₄alkoxy or phenyl substituents, it being possible for phenyl itself to be substituted by one or more halogen, C₁-C₄alkyl, C₁-C₄-haloalkyl, C₁-C₄alkoxy, —CN, —NO₂, C₁-C₄alkylthio, C₁-C₄alkylsulfinyl or C₁-C₄alkylsulfonyl substituents;

R₈ is hydrogen or C₁-C₈alkyl;

R₉ is hydrogen or C₁-C₈alkyl, or C₁-C₈alkyl substituted by one or more COOH, C₁-C₈-alkoxycarbonyl or —CN substituents, or

R₉ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or

R₈ and R₉ together denote C₂-C₅alkylene;

R₁₀ is hydrogen, C₁-C₄alkyl, C₁-C₄haloalkyl or C₃-C₆cycloalkyl;

R₁₁ is hydrogen, C₁-C₈alkyl, C₃-C₈alkenyl, C₃-C₈alkynyl, C₁-C₄haloalkyl or C₃-C₆haloalkenyl;

R₁₂ is hydrogen, C₁-C₄alkyl, C₁-C₄haloalkyl or C₃-C₆cycloalkyl;

R₁₃ is hydrogen, C₁-C₈alkyl, C₃-C₈alkenyl or C₃-C₈alkynyl; or

R₁₃ is phenyl or phenyl-C₁-C₆alkyl, it being possible for the phenyl ring itself to be substituted by one or more halogen, C₁-C₄alkyl, C₁-C₄haloalkyl, C₁-C₄alkoxy, —CN, —NO₂ or —S(O)₂C₁-C₈alkyl substituents, or

R₁₃ is C₁-C₈alkyl substituted by one or more halogen, —CN or C₁-C₄alkoxy substituents;

R₁₄ is hydrogen, C₁-C₈alkyl, C₃-C₈alkenyl or C₃-C₈alkynyl, or C₁-C₈alkyl substituted by one or more halogen, —CN or C₁-C₄alkoxy substituents;

R₁₅, R₁₆ and R₁₇ are each independently of the others C₁-C₈alkyl, C₃-C₈alkenyl or C₃-C₈-alkynyl, or C₁-C₈alkyl substituted by one or more halogen, —CN or C₁-C₄alkoxy substituents;

R₁₈ is hydrogen or C₁-C₈alkyl;

R₁₉ is hydrogen, C₁-C₈alkyl, C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkyl-sulfonyl substituents; or

R₁₈ and R₁₉ together denote a C₂-C₅alkylene chain, which may be interrupted by an oxygen or sulfur atom;

R₂₀ is hydrogen, C₁-C₈alkyl, C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkyl-sulfonyl substituents;

R₂₁ is hydrogen or C₁-C₈alkyl;

R₂₂ is hydrogen or C₁-C₈alkyl, or C₁-C₈alkyl substituted by one or more COOH, C₁-C₈-alkoxycarbonyl or —CN substituents, or

R₂₂ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or

R₂₁ and R₂₂together denote C₂-C₅alkylene;

R₂₃ is hydrogen, C₁-C₄alkyl, C₁-C₄haloalkyl or C₃-C₆cycloalkyl;

R₂₄ is hydrogen, C₁-C₄alkyl, C₁-C₄haloalkyl or C₃-C₆cycloalkyl;

R₂₅ is hydrogen, C₁-C₈alkyl, C₃-C₈alkenyl, C₃-C₈alkynyl, C₁-C₄haloalkyl or C₃-C₆haloalkenyl;

R₂₆ is hydrogen or C₁-C₈alkyl;

R₂₇ is hydrogen or C₁-C₈alkyl, or C₁-C₈alkyl substituted by one or more COOH, C₁-C₈-alkoxycarbonyl or —CN substituents, or

R₂₇ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or

R₂₆ and R₂₇together denote C₂-C₅alkylene;

R₂₈ is hydrogen or C₁-C₈alkyl;

R₂₉ and R₃₀ are each independently of the other hydrogen, C₁-C₈alkyl, C₃-C₈alkenyl or C₃-C₈alkynyl, or C₁-C₈alkyl substituted by one or more halogen, —CN or C₁-C₄alkoxy substituents;

R₃₁ and R₃₂ are each independently of the other C₁-C₈alkyl, C₃-C₈alkenyl or C₃-C₈alkynyl, or C₁-C₈alkyl substituted by one or more halogen, —CN or C₁-C₄alkoxy substituents;

m is 0, 1, 2, 3, 4 or 5;

each R₂ independently of any other(s) is halogen, —CN, —SCN, —SF₅, —NO₂, —NR₃₆R₃₇, —CO₂R₃₈, —CONR₃₉R₄₀, —C(R₄₁)═NOR₄₂, —COR₄₃, —OR₄₄, —SR₄₅, —SOR₄₆, —SO₂R₄₇, OSO₂R₄₈—N([CO]ₚR₄₉)COR₅₀, —N(OR₅₁)COR₅₂, —N(R₅₃)CO₂R₅₄ or —N-phthalimide;

R₃₆ is hydrogen or C₁-C₈alkyl; and

R₃₇ is hydrogen, C₁-C₈alkyl, C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄alkoxy, —CN, —NO₂, C_{-C}₄alkylthio, C₁-C₄alkylsulfinyl or C₁-C₄alkyl-sulfonyl substituents; or

R₃₆ and R₃₇ together denote a C₂-C₅alkylene chain, which may be interrupted by an oxygen or sulfur atom;

R₃₈ is hydrogen, C₁-C₈alkyl, C₃-C₈alkenyl or C₃-C₈alkynyl, or C₁-C₈alkyl, C₃-C₈alkenyl or C₃-C₈alkynyl substituted by one or more halogen, C₁-C₄alkoxy or phenyl substituents, it being possible for phenyl itself to be substituted by one or more halogen, C₁-C₄alkyl, C₁-C₄-haloalkyl, C₁-C₄alkoxy, —CN, —NO₂, C₁-C₄alkylthio, C₁-C₄alkylsulfinyl or C₁-C₄alkylsulfonyl substituents;

R₃₉ is hydrogen or C₁-C₈alkyl;

R₄₀ is hydrogen or C₁-C₈alkyl, or C₁-C₈alkyl substituted by one or more —COOH, C₁-C₈-alkoxycarbonyl or —CN substituents, or

R₄₀ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or

R₃₉and R₄₀together denote C₃-C₅alkylene;

R₄₁ is hydrogen, C₁-C₄alkyl, C₁-C₄haloalkyl or C₃-C₆cycloalkyl;

R₄₂ is hydrogen, C₁-C₈alkyl, C₃-C₈alkenyl, C₃-C₈alkynyl, C₁-C₄haloalkyl or C₃-C₆haloalkenyl;

R₄₃ is hydrogen, C₁-C₄alkyl, C₁-C₄haloalkyl or C₃-C₆cycloalkyl;

R₄₄ is hydrogen, C₁-C₈alkyl, C₃-C₈alkenyl or C₃-C₈alkynyl; or

R₄₄ is phenyl or phenyl-C₁-C₆alkyl, it being possible for the phenyl ring itself to be substituted by one or more halogen, C₁-C₄alkyl, C₁-C₄haloalkyl, C₁-C₄alkoxy, —CN, —NO₂ or —S(O)₂C₁-C₈alkyl substituents, or

R₄₄ is C₁-C₈alkyl substituted by one or more halogen, —CN or C₁-C₄alkoxy substituents;

R₄₅ is hydrogen, C₁-C₈alkyl, C₃-C₈alkenyl or C₃-C₈alkynyl, or C₁-C₈alkyl substituted by one or more halogen, —CN or C₁-C₄alkoxy substituents;

R₄₆, R₄₇ and R₄₈ are each independently of the others C₁-C₈alkyl, C₃-C₈alkenyl or C₃-C₈-alkynyl, or C₁-C₈alkyl substituted by one or more halogen, —CN or C₁-C₄alkoxy substituents;

p is 0 or 1;

R₄₉, R₅₀, R₅₁, R₅₂, R₅₃ and R₅₄ are each independently of the others hydrogen, C₁-C₈alkyl, or phenyl, which may itself be substituted by one or more halogen, C₁-C₈alkyl, C₁-C₄haloalkyl, C₁-C₄alkoxy, —CN, —NO₂, C₁-C₈alkylthio, C₁-C₈alkylsulfinyl or C₁-C₈alkylsulfonyl substituents; or

each R₂ independently of any other(s) is C₁-C₈alkyl, or C₁-C₈alkyl mono- or poly-substituted by halogen, —CN, —NO₂, —NR₅₅R₅₆, —CO₂R₅₇, —CONR₅₈R₅₉, —COR₆₀, —C(R₆₁)═NOR₆₂, —C(S)NR₆₃R₆₄, —C(C₁-C₄alkylthio)=NR₆₅, —OR₆₆, —SR₆₇, —SOR₆₈, —SO₂R₆₉, —O(SO₂)R₇₀, —N(R₇₁)CO₂R₇₂, —N(R₇₃)COR₇₄ or by C₃-C₆cycloalkyl; or

each R₂independently of any other(s) is C₂-C₈alkenyl, or C₂-C₈alkenyl mono- or poly-substituted by —CN, —NO₂, —CO₂R₇₅, —CONR₇₆R₇₇, —COR₇₈, —C(R₇₉)═NOR₈₀, —C(S)NR₈₁R₈₂, —C(C₁-C₄alkylthio)=NR₈₃ or by C₃-C₆cycloalkyl; or

each R₂ independently of any other(s) is C₂-C₈alkynyl, or C₂-C₈alkynyl mono- or poly-substituted by halogen, —CN, —CO₂R₈₄, —CONR₈₅R₈₆, —COR₈₇, —C(R₈₈)═NOR₈₉, —C(S)NR₉₀R₉₁, —C(C₁-C₄alkylthio)=NR₉₂ or by C₃-C₆cycloalkyl; or

each R₂ independently of any other(s) is C₃-C₆cycloalkyl, or C₃-C₆cycloalkyl mono- or poly-substituted by halogen, —CN, —CO₂R₉₃, —CONR₉₄R₉₅, —COR₉₆, —C(R₉₇)═NOR₉₈, —C(S)NR₉₉R₁₀₀ or by —C(C₁-C₄alkylthio)=NR₁₀₁; or

two adjacent R₂ together form a C₁-C₇alkylene bridge, which may be interrupted by 1 or 2 non-adjacent oxygen atoms and may be substituted by C₁-C₆alkyl, the total number of ring atoms being at least 5 and a maximum of 9; or two adjacent R₂ together form a C₂-C₇-alkenylene bridge, which may be interrupted by 1 or 2 non-adjacent oxygen atoms and may be substituted by C₁-C₆alkyl, the total number of ring atoms being at least 5 and a maximum of 9;

R₅₅ is hydrogen or C₁-C₈alkyl;

R₅₆ is hydrogen, C₁-C₈alkyl, C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkyl-sulfonyl substituents; or

R₅₅ and R₅₆ together denote a C₂-C₅alkylene chain, which may be interrupted by an oxygen or sulfur atom;

R₅₇ is hydrogen, C₁-C₈alkyl, C₃-C₈alkenyl or C₃-C₈alkynyl, or C₁-C₈alkyl, C₃-C₈alkenyl or C₃-C₈alkynyl substituted by one or more halogen, C₁-C₄alkoxy or phenyl substituents, it being possible for phenyl itself to be substituted by one or more halogen, C₁-C₄alkyl, C₁-C₄-haloalkyl, C₁-C₄alkoxy, —CN, —NO₂, C₁-C₄alkylthio, C_{1-C}₄alkylsulfinyl or C₁-C₄alkylsulfonyl substituents;

R₅₈ is hydrogen or C₁-C₈alkyl;

R₅₉ is hydrogen or C₁-C₈alkyl, or C₁-C₈alkyl substituted by one or more —COOH, C₁-C₈-alkoxycarbonyl or —CN substituents; or

R₅₉ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or

R₅₈ and R₅₉ together denote C₂-C₅alkylene;

R₆₀ is hydrogen, C₁-C₄alkyl, C₁-C₄haloalkyl or C₃-C₆cycloalkyl;

R₆₁ is hydrogen, C₁-C₄alkyl, C₁-C₄haloalkyl or C₃-C₆cycloalkyl;

R₆₂ is hydrogen, C₁-C₈alkyl, C₃-C₈alkenyl, C₃-C₈alkynyl, C₁-C₄haloalkyl or C₃-C₆-haloalkenyl; and

R₆₃ is hydrogen or C₁-C₈alkyl;

R₆₄ is hydrogen or C₁-C₈alkyl, or C₁-C₈alkyl substituted by one or more —COOH, C₁-C₈-alkoxycarbonyl or —CN substituents; or

R₆₄ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or

R₆₃ and R₆₄ together denote C₂-C₅alkylene;

R₆₅ is hydrogen or C₁-C₈alkyl;

R₆₆ and R₆₇ are each independently of the other hydrogen, C₁-C₈alkyl, C₃-C₈alkenyl, C₃-C₈-alkynyl, or C₁-C₈alkyl substituted by one or more halogen, —CN or C₁-C₄alkoxy substituents;

R₆₈, R₆₉ and R₇₀ are each independently of the others C₁-C₈alkyl, C₃-C₈alkenyl or C₃-C₈-alkynyl, or C₁-C₈alkyl substituted by one or more halogen, —CN or C₁-C₄alkoxy substituents;

R₇₁ and R₇₃ are each independently of the other hydrogen, C₁-C₈alkyl or C₁-C₈alkoxy;

R₇₂ is C₁-C₈alkyl;

R₇₄ is hydrogen or C₁-C₈alkyl;

R₇₅ is hydrogen, C₁-C₈alkyl, C₃-C₈alkenyl or C₃-C₈alkynyl, each of which may be mono- or poly-substituted by one or more halogen, C₁-C₄alkoxy or phenyl substituents, it being possible for phenyl itself to be substituted by one or more halogen, C₁-C₄alkyl, C₁-C₄-haloalkyl, C₁-C₄alkoxy, —CN, —NO₂, C₁-C₄alkylthio, C₁-C₄alkylsulfinyl or C₁-C₄alkylsulfonyl substituents;

R₇₆ is hydrogen or C₁-C₈alkyl;

R₇₇ is hydrogen or C₁-C₈alkyl, or C₁-C₈alkyl substituted by one or more —COOH, C₁-C₈-alkoxycarbonyl or CN substituents; or

R₇₇ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or

R₇₆ and R₇₇ together denote C₂-C₅alkylene;

R₇₈ and R₇₉ are each independently of the other hydrogen, C₁-C₄alkyl, C₁-C₄haloalkyl or C₃-C₆cycloalkyl;

R₈₀ is hydrogen, C₁-C₈alkyl, C₃-C₈alkenyl, C₃-C₈alkynyl, C₁-C₄haloalkyl or C₃-C₆haloalkenyl;

R₈₁ is hydrogen or C₁-C₈alkyl;

R₈₂ is hydrogen or C₁-C₈alkyl, or C₁-C₈alkyl substituted by one or more —COOH, C₁-C₈-alkoxycarbonyl or —CN substituents; or

R₈₂ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or

R₈₁ and R₈₂together denote C₂-C₅alkylene;

R₈₃ is hydrogen or C₁-C₈alkyl;

R₈₄ is hydrogen, C₁-C₈alkyl, C₃-C₈alkenyl or C₃-C₈alkynyl, each of which may be mono- or poly-substituted by one or more halogen, C₁-C₄alkoxy or phenyl substituents, it being possible for phenyl itself to be substituted by one or more halogen, C₁-C₄alkyl, C₁-C₄-haloalkyl, C₁-C₄alkoxy, —CN, —NO₂, C₁-C₄alkylthio, C₁-C₄alkylsulfinyl or C₁-C₄alkylsulfonyl substituents;

R₈₅ is hydrogen or C₁-C₈alkyl;

R₈₆ is hydrogen or C₁-C₈alkyl, or C₁-C₈alkyl substituted by one or more —COOH, C₁-C₈-alkoxycarbonyl or —CN substituents; or

R₈₆ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or

R₈₅and R₈₆together denote C₂-C₅alkylene;

R₈₇ is hydrogen, C₁-C₄alkyl, C₁-C₄haloalkyl or C₃-C₆cycloalkyl;

R₈₈ is hydrogen, C₁-C₄alkyl, C₁-C₄haloalkyl or C₃-C₆cycloalkyl;

R₈₉ is hydrogen, C₁-C₈alkyl, C₃-C₈alkenyl, C₃-C₈alkynyl, C₁-C₄haloalkyl or C₃-C₆haloalkenyl;

R₉₀ is hydrogen or C₁-C₈alkyl;

R₉₁ is hydrogen or C₁-C₈alkyl, or C₁-C₈alkyl substituted by one or more —COOH, C₁-C₈-alkoxycarbonyl or —CN substituents; or

R₉₁ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or

R₉₀ and R₉₁ together denote C₂-C₅alkylene;

R₉₂ is hydrogen or C₁-C₈alkyl;

R₉₃ is hydrogen, C₁-C₈alkyl, C₃-C₈alkenyl or C₃-C₈alkynyl, each of which may be mono- or poly-substituted by one or more halogen, C₁-C₄alkoxy or phenyl substituents, it being possible for phenyl itself to be substituted by one or more halogen, C₁-C₄alkyl, C₁-C₄-haloalkyl, C₁-C₄alkoxy, —CN, —NO₂, C₁-C₄alkylthio, C₁-C₄alkylsulfinyl or C_{1-C}₄alkylsulfonyl substituents;

R₉₄ is hydrogen or C₁-C₈alkyl;

R₉₅ is hydrogen or C₁-C₈alkyl, or C₁-C₈alkyl substituted by one or more —COOH, C₁-C₈-alkoxycarbonyl or —N substituents; or

R₉₅ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or

R₉₄and R₉₅ together denote C₂-C₅alkylene;

R₉₆ is hydrogen, C₁-C₄alkyl, C₁-C₄haloalkyl or C₃-C₆cycloalkyl;

R₉₇ is hydrogen, C₁-C₄alkyl, C₁-C₄haloalkyl or C₃-C₆cycloalkyl;

R₉₈ is hydrogen, C₁-C₈alkyl, C₃-C₈alkenyl, C₃-C₈alkynyl, C₁-C₄haloalkyl or C₃-C₆haloalkenyl;

R₉₉ is hydrogen or C₁-C₈alkyl;

R₁₀₀ is hydrogen or C₁-C₈alkyl, or C₁-C₈alkyl substituted by one or more —COOH, C₁-C₈-alkoxycarbonyl or —CN substituents; or

R₁₀₀ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or

R₉₉ and R₁₀₀together denote C₂-C₅alkylene; and

R₁₀₁ is hydrogen or C₁-C₈alkyl,

and to agrochemically acceptable salts and all stereoisomers and tautomers of the compounds of formula I.

When n is 0, all the free valences on the pyridine ring of the compounds of formula I are substituted by hydrogen. When m is 0, all the free valences on the phenyl ring of the compounds of formula I are substituted by hydrogen.

Examples of substituents that are formed when R₅ and R₆ together or R₁₈ and R₁₉ together or R₃₆ and R₃₇ together or R₅₅ and R₅₆ together denote a C₂-C₅alkylene chain, which may be interrupted by an oxygen or sulfur atom, are piperidine, morpholine, thiomorpholine and pyrrolidine.

The alkyl groups occurring in the definitions of substituents may be straight-chain or branched and are, for example, methyl, ethyl, n-propyl, isopropyl, n-butyl, sec-butyl, isobutyl or tert-butyl, and the pentyl, hexyl, heptyl, octyl, nonyl and decyl isomers.

Halogen is fluorine, chlorine, bromine or iodine, preferably fluorine or chlorine. Haloalkyl is, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl or 2,2,2-trichloroethyl; preferably trichloromethyl, difluorochloromethyl, difluoromethyl, trifluoromethyl or dichlorofluoromethyl.

Alkoxy groups preferably have a chain length of from 1 to 6, especially from 1 to 4, carbon atoms. Alkoxy is, for example, methoxy, ethoxy, propoxy, isopropoxy, n-butoxy, isobutoxy, sec-butoxy or tert-butoxy, and the pentyloxy and hexyloxy isomers; preferably methoxy or ethoxy.

Alkoxy, alkenyl, alkynyl, alkoxyalkyl, alkylthio, alkylsulfonyl, alkylsulfinyl, alkylaminoalkoxy, alkoxycarbonyl, alkylcarbonyloxy, alkenylthio, alkenylsulfonyl, alkenylsulfinyl, alkynyl-sulfonyl, alkynylthio and alkynylsulfinyl groups are derived from the mentioned alkyl radicals. The alkenyl and alkynyl groups may be mono- or poly-unsaturated. Alkenyl is to be understood as meaning, for example, vinyl, allyl, methallyl, 1-methylvinyl or but-2-en-1-yl. Alkynyl is, for example, ethynyl, propargyl, but-2-yn-1-yl, 2-methylbutyn-2-yl or but-3-yn-2-yl.

Alkylthio groups preferably have a chain length of from 1 to 4 carbon atoms. Alkylthio is, for example, methylthio, ethylthio, propylthio, isopropylthio, n-butylthio, isobutylthio, sec-butyl-thio or tert-butylthio, preferably methylthio or ethylthio. Alkylsulfinyl is, for example, methylsulfinyl, ethylsulfinyl, propylsulfinyl, isopropylsulfinyl, n-butylsulfinyl, isobutylsulfinyl, sec-butylsulfinyl or tert-butylsulfinyl; preferably methylsulfinyl or ethylsulfinyl. Alkylsulfonyl is, for example, methylsulfonyl, ethylsulfonyl, propylsulfonyl, isopropylsulfonyl, n-butylsulfonyl, isobutylsulfonyl, sec-butylsulfonyl or tert-butylsulfonyl; preferably methylsulfonyl or ethyl-sulfonyl.

Alkoxyalkyl groups preferably have from 1 to 6 carbon atoms. Alkoxyalkyl is, for example, methoxymethyl, methoxyethyl, ethoxymethyl, ethoxyethyl, n-propoxymethyl, n-propoxyethyl, isopropoxymethyl or isopropoxyethyl.

Substituents wherein two adjacent R₁ together form a C₁-C₇alkylene bridge, which may be interrupted by 1 or 2 non-adjacent oxygen atoms and may be substituted by C₁-C₆alkyl, the total number of ring atoms being at least 5 and a maximum of 9; or wherein two adjacent R₁ together form a C₂-C₇alkenylene bridge, which may be interrupted by 1 or 2 non-adjacent oxygen atoms and may be substituted by C₁-C₆alkyl, the total number of ring atoms being at least 5 and a maximum of 9, have, for example, the following structures:

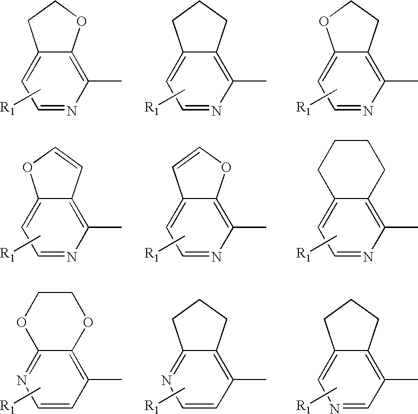

Substituents wherein two adjacent R₂ together form a C₁-C₇alkylene bridge, which may be interrupted by 1 or 2 non-adjacent oxygen atoms and may be substituted by C₁-C₆alkyl, the total number of ring atoms being at least 5 and a maximum of 9; or wherein two adjacent R₂ together form a C₂-C₇alkenylene bridge, which may be interrupted by 1 or 2 non-adjacent oxygen atoms and may be substituted by C₁-C₆alkyl, the total number of ring atoms being at least 5 and a maximum of 9, have, for example, the following structures:

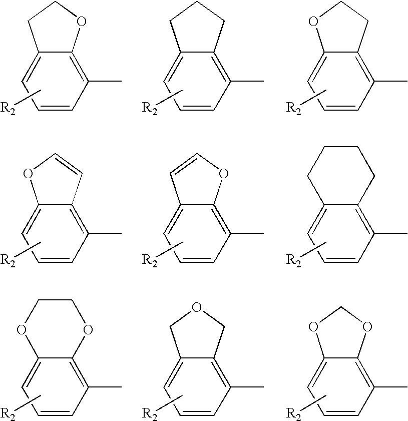

The invention also includes the salts that the compounds of formula I are able to form preferably with amines, alkali metal and alkaline earth metal bases or quaternary ammonium bases. Suitable salt formers are described, for example, in WO 98/41089.

Of the alkali metal and alkaline earth metal hydroxides as salt formers, special mention may be made of the hydroxides of lithium, sodium, potassium, magnesium or calcium, but especially those of sodium or potassium.

As examples of amines suitable for the formation of ammonium salts, there come into consideration both ammonia and primary, secondary and tertiary C₁-C₁₈alkylamines, C₁-C₄-hydroxyalkylamines and C₂-C₄alkoxyalkylamines, for example methylamine, ethylamine, n-propylamine, isopropylamine, the four isomeric butylamines, n-amylamine, isoamylamine, hexylamine, heptylamine, octylamine, nonylamine, decylamine, pentadecylamine, hexadecylamine, heptadecylamine, octadecylamine, methyl-ethylamine, methyl-iso-propylamine, methyl-hexylamine, methyl-nonylamine, methyl-pentadecylamine, methyl-octadecylamine, ethyl-butylamine, ethyl-heptylamine, ethyl-octylamine, hexyl-heptylamine, hexyl-octylamine, dimethylamine, diethylamine, di-n-propylamine, diisopropylamine, di-n-butylamine, di-n-amylamine, diisoamylamine, dihexylamine, diheptylamine, dioctylamine, ethanolamine, n-propanolamine, isopropanolamine, N,N-diethanolamine, N-ethylpropanol-amine, N-butylethanolamine, allylamine, n-butenyl-2-amine, n-pentenyl-2-amine, 2,3-dimethylbutenyl-2-amine, dibutenyl-2-amine, n-hexenyl-2-amine, propylenediamine, trimethylamine, triethylamine, tri-n-propylamine, triisopropylamine, tri-n-butylamine, triiso-butylamine, tri-sec-butylamine, tri-n-amylamine, methoxyethylamine and ethoxyethylamine; heterocyclic amines, e.g. pyridine, quinoline, isoquinoline, morpholine, piperidine, pyrrolidine, indoline, quinuclidine and azepine; primary arylamines, e.g. anilines, methoxyanilines, ethoxyanilines, o-, m- and p-toluidines, phenylenediamines, benzidines, naphthylamines and o-, m- and p-chloroanilines; but especially triethylamine, isopropylamine and diisopropylamine.

Preferred quarternary ammonium bases that are suitable for salt formation correspond, for example, to the formula [N(RₐR_{b}R_{c}R_{d})]OH, wherein Rₐ, R_{b}, R_{c} and R_{d} are each independently of the others C₁-C₄alkyl. Other suitable tetraalkylammonium bases with other anions can be obtained, for example, by anion exchange reactions.

Preferred compounds of formula I are those wherein each R₁ independently of any other(s) is halogen, —CN, —NO₂, —C(R₁₀)═NOR₁₁, —OR₁₃, —SO₂R₁₆, —OSO₂R₁₇, C₁-C₈alkyl or C₂-C₈alkenyl, or C₁-C₈alkyl substituted by one or more halogen or —CN substituents;

R₁₀ is hydrogen or C₁-C₄alkyl; and

R₁₁ is C₁-C₈alkyl.

Preference is given also to those compounds of formula I wherein each R₂ independently of any other(s) is halogen, —CN, —NO₂, —NR₃₆R₃₇, —CO₂R₃₈, —C(R₄₁)═NOR₄₂, —OR₄₄, —SO₂R₄₇, —OSO₂R₄₈, C₁-C₈alkyl, or C₁-C₈alkyl mono- or poly-substituted by halogen, —CN or by 13 CO₂R₅₇;

R₃₆ and R₃₇ are hydrogen;

R₃₈ is hydrogen or C₁-C₈alkyl;

R₄₁ is hydrogen or C₁-C₄alkyl; and

R₄₂ is C₁-C₈alkyl.

In an especially preferred group of compounds of formula I, each R₁ independently of any other(s) is halogen, —CN, —NO₂, —C(R₁₀)═NOR₁₁, —OR₁₃, —SO₂R₁₆, —OSO₂R₁₇, C₁-C₈alkyl or C₂-C₈alkenyl, or C₁-C₈alkyl substituted by one or more —CN substituents;

R₁₀ is hydrogen or C₁-C₄alkyl;

R₁₁ is C₁-C₈alkyl;

each R₂ independently of any other(s) is halogen, —CN, —NO₂, —NR₃₆R₃₇, —CO₂R₃₈, —C(R₄₁)═NOR₄₂, —OR₄₄, —SO₂R₄₇, —OSO₂R₄₈ or C₁-C₈alkyl, or C₁-C₈alkyl mono- or poly-substituted by —CN or by —CO₂R₅₇;

R₃₆ and R₃₇ are hydrogen;

R₃₈ is hydrogen or C₁-C₈alkyl;

R₄₁ is hydrogen or C₁-C₄alkyl;

R₄₂ is C₁-C₈alkyl; and

R₃ and R₄ are each independently of the other hydrogen or C₁-C₄alkyl.

Also of interest are compounds of formula I wherein R₁ is halogen, —CN, C₁-C₈alkyl substituted by —CN, or C₁-C₈alkoxy.

Very special preference is given to those compounds of formula I wherein R₂ is halogen, —CN, C₁-C₈alkyl substituted by —CN, or C₁-C₈alkoxy, at least one of the substituents R₁ and R₂ being especially C₁-C₈alkyl substituted by —CN.

Preference is given also to compounds of formula I wherein n is 0, 1 or 2, and m is 0, 1, 2, 3 or 4, n being especially 1 or 2, and m being especially 1 or 2.

Of particular interest are compounds of formula I wherein R₃ and R₄ are hydrogen.

In an outstanding group of compounds of formula I, the group

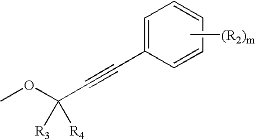

occupies the 2-position on the pyridine ring, n being especially 1 or 2, and R₁ occupying especially the 3- and/or 5-position on the pyridine ring.

Preference is given also to compounds of formula I wherein m is 1 or 2, and R₂ occupies the 3-position on the phenyl ring.

Special mention may be made also of compounds of formula I, wherein R₁ is hydrogen, fluorine, chlorine, bromine, methoxy, difluoromethoxy, trifluoromethyl or isopropylthio;

R₂ is cyanomethyl, chlorine or bromine;

R₃ and R₄ are hydrogen;

n is 1 or 2, and

m is 1.

The compounds of formula I can be prepared by processes known per se described, for example, in J. Chem. Soc. Perkin Trans. 1979, pages 2756-2761; Synth. Commun. 1988, 18, pages 1111-1118; J. Org Chem. 1996, 61, pages 4258-4261; and K. Sonogashira, Comprehensive Organic Synthesis 1991, Vol. 3, page 521, for example, by reacting a compound of formula II

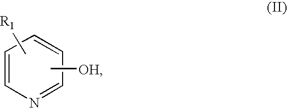

wherein R₁ is as defined for formula I, in the presence of a base, with a compound of formula III

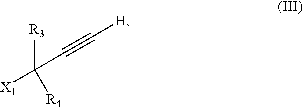

wherein R₃ and R₄ are as defined for formula I and X₁ is O-tosyl, chlorine, bromine or iodine, to form a compound of formula IV

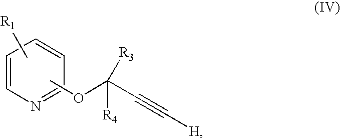

wherein R₁, R₃ and R₄ are as defined for formula I, and then coupling that compound, in the presence of a palladium catalyst, with a compound of formula V

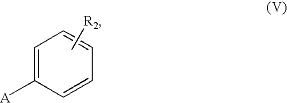

wherein R₂ is as defined for formula I and A is a leaving group, such as halogen or trifluoromethanesulfonate.

The preparation of the compounds of formula I can be carried out, for example, in accordance with Reaction Schemes 1, 2, 3,4, 5 and 6. For the individual synthesis schemes, it is generally the case that different R₁ substituents may already be present at the start, or can be introduced in succession, for example by nucleophilic substitution.

According to Reaction Scheme 1, the compounds of formula I can be obtained, for example, from substituted pyridyl propargyl ethers of formula IV.

The propargyl ethers of formula IV can be obtained in advance by alkylation of hydroxypyridines of formula II, which are reacted with acetylene derivatives III in the presence of a base. Such alkylations are standard procedures and can be carried out, for example, analogously to J. Chem. Soc. Perkin Trans. 1979, pages 2756-2761; Synth. Commun. 1988, 18, pages 1111-1118; and J. Org Chem. 1996, 61, pages 4258-4261.

In the next Step, the propargyl ethers of formula IV are coupled with substituted aryls of formula V under typical Sonogashira conditions (K. Sonogashira, Comprehensive Organic Synthesis 1991, Vol. 3, page 521). As catalyst mixtures there come into consideration, for example, tetrakistriphenylphosphinepalladium or bistriphenylphosphinepalladium dichloride together with copper iodide; as bases (for the reductive elimination) there come into consideration preferably amines, for example triethylamine, diethylamine or diisopropyl-ethylamine.

The aryls of formula V preferably carry a leaving group A, wherein A is, for example, halogen (N. Krause et al., J. Org. Chem. 1998, 63, page 8551; and Nakamura, H. et al., Tetrahedron Lett. 2000, 41, page 2185) or trifluoromethanesulfonate (Ritter, K., Synthesis 1993, page 735). As solvents there are customarily used ethers, for example tetra-hydrofuran, chlorinated hydrocarbons, for example chloroform, or dipolar aprotic solvents, for example dimethylformamide or dimethyl sulfoxide.

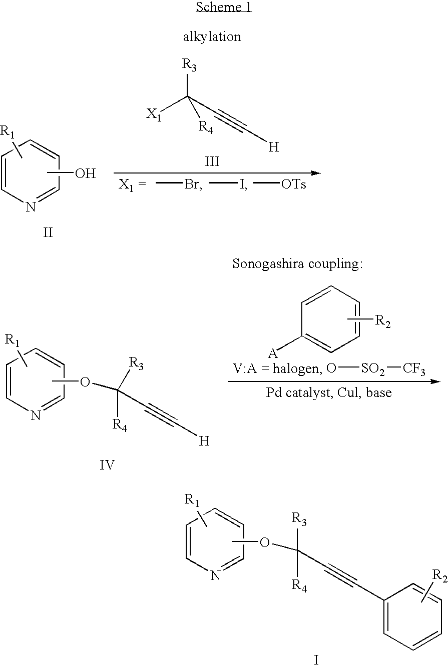

The Pd-catalysed cross-coupling of suitably substituted benzenes of formula V with propargyl alcohols or terminal acetylenes of formula VI is generally known as a Sonogashira reaction (Reaction Scheme 2). That reaction has already been depicted in detail (see above, Scheme 1) and can also be used for the preparation of the phenylpropargyl alcohols of formula VII.

The activation of the alcohol of formula VII is achieved, for example, by tosylation or halogenation. The tosylation of the alcohol of formula VII is a standard reaction and can be carried out, for example, with a sulfonic acid chloride, for example mesyl chloride or para-toluenesulfonic acid chloride (p-TosCl) in the presence of an amine, for example diethyl-amine, triethylamine or pyridine, in a solvent, e.g. a chlorinated hydrocarbon, for example carbon tetrachloride or methylene chloride, or an amine, for example pyridine. Such reactions are generally known and are described, for example, in J. Org. Chem. 1997, 62, page 8987; J. Het. Chem. 1995, 32, pages 875-882; and Tetrahedron Lett. 1997, 38, pages 8671-8674.

The halogenation can be carried out analogously to standard procedures. For example, bromination is effected using carbon tetrabromide in the presence of triphenylphosphine (Synthesis 1998, pages 1015-1018) in methylene chloride. Chlorination is effected using mineral acids, for example using concentrated hydrochloric acid (J. Org. Chem. 1955, 20, page 95) or using para-toluenesulfonic acid chloride in the presence of an amine, for example triethylamine, in a solvent, for example methylene chloride (Tetrahedron Lett. 1984, 25, page 2295).

The preparation of the phenyl-propynyloxy-pyridines of formula I can be carried out analogously to Synthesis 1995, pages 707-712; and Tetrahedron Lett. 1994, 35, pages 6405-6408 by means of copper iodide-catalysed alkylation. Suitable solvents are dimethylformamide and acetonitrile; suitable bases are preferably potassium carbonate and 1,8-diazabicylo[5.4.0]-undec-7-ene (DBU).

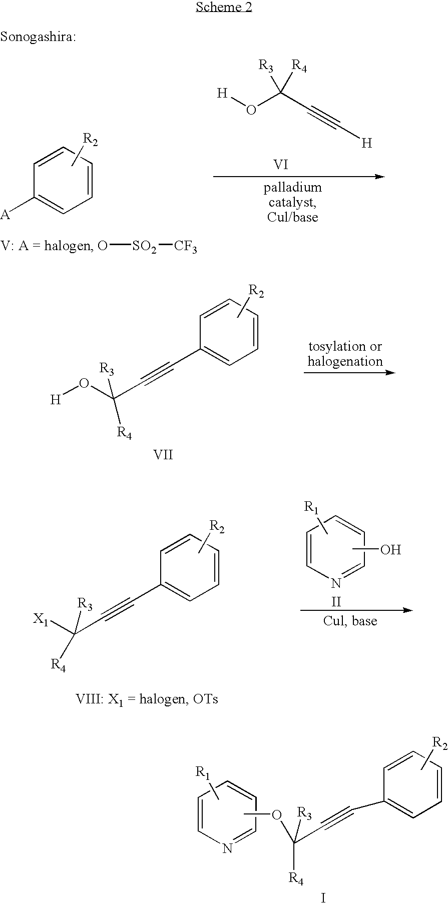

Compounds of formula I can also be obtained according to other methods (see Scheme 3).

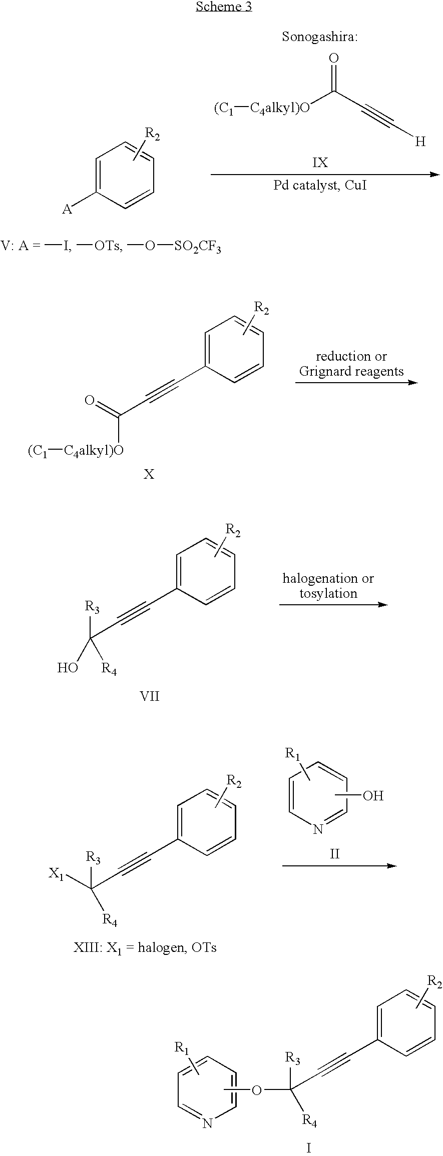

Phenylacetylene esters of formula X can be obtained by means of Sonogashira coupling from the compounds of formula IX and activated benzene derivatives of formula V. The esters of formula X can then be reduced or reacted with organometal compounds, for example Grignard reagents, to form the alcohols of formula VII.

The reduction can be carried out preferably with hydrides according to standard methods, for example with lithium aluminium hydride or sodium borohydride in a solvent, e.g. an ether, for example diethyl ether, dioxane or tetrahydrofuran, or an alcohol, for example methanol or ethanol. Such reductions are described, for example, in C. Ferri, “Reaktionen der organischen Synthese” 1978, pages 98-102.

Reactions of carboxylic acid esters with Grignard reagents are standard in organic synthesis chemistry and are described in detail, for example, in “Organikum” 1976, pages 617-625. The subsequent etherification of the pyridyl derivatives of formula II to form the compounds of formula I has already been depicted in detail in Scheme 2.

Further methods of preparing the compounds of type I are shown in Scheme 4 (variant of Scheme 3).

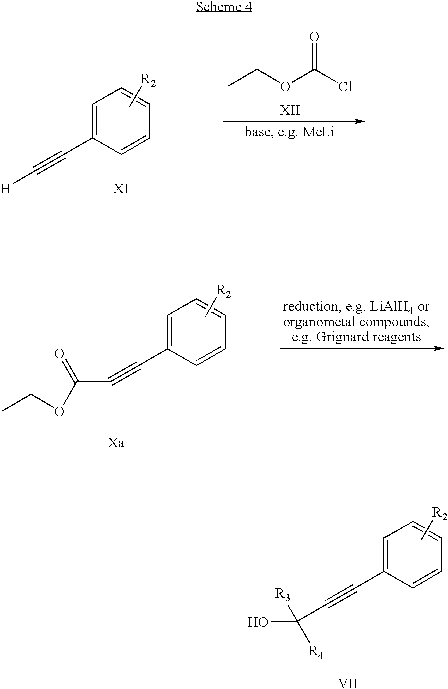

The reaction of phenylacetylenes of formula XI with methyllithium and subsequent reaction with chloroformic acid ethyl ester of formula XII yields the ester of formula Xa, which can be converted to the compounds of type I via an alcohol of formula VII in a manner completely analogous to that already shown in Scheme 3 (Tetrahedron. Left. 1992, 33, page 4495).

The nucleophilic aromatic substitution of the pyridine derivatives of formula XIV, wherein X₂ is halogen (Reaction Scheme 5) can be carried out analogously to known procedures, as described, for example, in J. March, “Advanced Organic Chemistry” 4th Edition, John Wiley & Sons, New York, 1992, pages 641-676. Accordingly the pyridine derivative of formula XIV is reacted with a propargyl alcohol of formula VII in an aprotic solvent, e.g. an amide, for example N,N-dimethylformamide (DMF) or 1-methyl-2-pyrrolidone (NMP), a sulfoxide, for example dimethyl sulfoxide (DMSO), a ketone, for example acetone, or an ether, for example tetrahydrofuran (THF), in the presence of a base, e.g. a carbonate, for example potassium or caesium carbonate, or a metal hydride, for example sodium hydride, at temperatures of from 0° C. to 100° C. (see also EP-A-0 759 429).

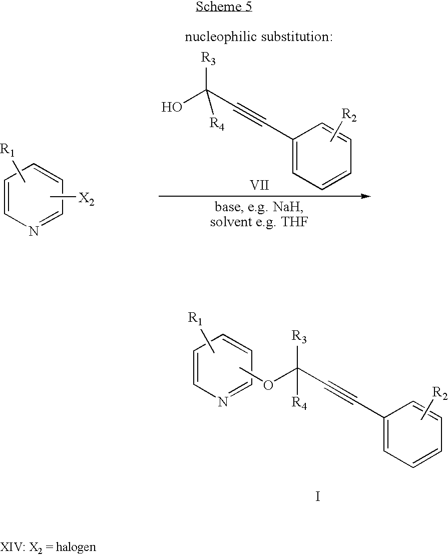

Compounds of formula I can also be prepared by first reacting the propargyl alcohols of formula XV with the pyridine derivatives of formula XIV to form compounds of formula XVI and only then in the next synthesis step carrying out a Sonogashira reaction with activated benzene derivatives of formula V (Reaction Scheme 6).

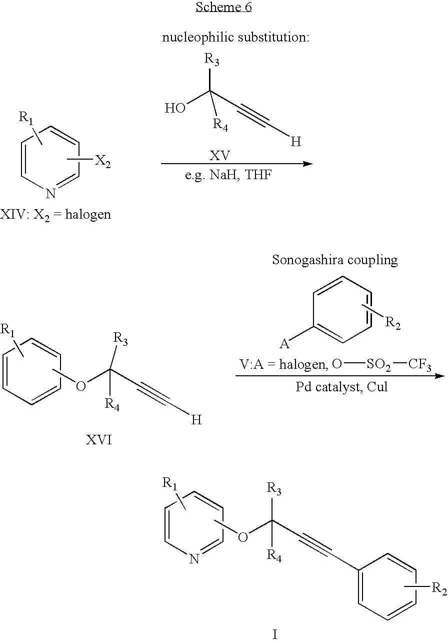

For the individual reaction steps (Schemes 1 to 6) the following applies: The reactions to form compounds of formula I are advantageously carried out in aprotic, inert organic solvents. Such solvents are hydrocarbons, such as benzene, toluene, xylene or cyclohexane, chlorinated hydrocarbons, such as dichloromethane, trichloromethane, tetrachloromethane or chlorobenzene, ethers, such as diethyl ether, ethylene glycol dimethyl ether, diethylene glycol dimethyl ether, tetrahydrofuran or dioxane, nitrites, such as acetonitrile or propionitrile, or amides, such as N,N-dimethylformamide, diethylformamide or N-methylpyrrolidinone. The reaction temperatures are preferably from −20° C. to +120° C. The reactions are generally slightly exothermic and can usually be carried out at room temperature. In order to shorten the reaction time or to initiate the reaction, it is optionally possible to heat the reaction mixture for a short time up to boiling point. The reaction times can also be reduced by the addition of a few drops of base as reaction catalyst. Suitable bases are especially tertiary amines, such as trimethylamine, triethylamine, quinuclidine, 1,4-diazabicyclo[2.2.2]octane, 1,5-diazabicyclo[4.3.0]non-5-ene or 1,5-diazabicyclo[5.4.0]-undec-7-ene. The bases used can, however, also be inorganic bases, such as hydrides, such as sodium or calcium hydride, hydroxides, such as sodium or potassium hydroxide, carbonates, such as sodium or potassium carbonate, or hydrogen carbonates, such as potassium or sodium hydrogen carbonate.

The compounds of formula I can be isolated in customary manner by concentration and/or evaporation of the solvent and can be purified by recrystallisation or trituration of the solid residue in solvents in which they are not readily soluble, such as ethers, aromatic hydrocarbons or chlorinated hydrocarbons.

For the use of the compounds of formula I according to the invention or compositions comprising them, there are suitable any of the methods of application customary in agriculture, such as preemergence application, postemergence application and seed dressing, as well as various methods and techniques, such as the controlled release of active ingredient. In the latter method, the compound is applied in solution to mineral granule carriers or polymerised granules (urea/formaldehyde) and dried. Where appropriate, it is possible in addition to apply a coating (coated granules) which allows the active ingredient to be released in metered amounts over a specific period.

The compounds of formula I can be used as herbicides in unmodified form, i.e. as obtained during synthesis, but are preferably formulated in customary manner together with the adjuvants conventionally employed in formulation technology, e.g. into emulsifiable concentrates, directly sprayable or dilutable solutions, dilute emulsions, wettable powders, soluble powders, dusts, granules and microcapsules. Such formulations are described, for example, in WO 97/34485 on pages 9 to 13. As with the nature of the compositions, the methods of application, such as spraying, atomising, dusting, wetting, scattering or pouring, are chosen in accordance with the intended objectives and the prevailing circumstances.

The formulations, i.e. the compositions, preparations or mixtures comprising the compound of formula I or at least one compound of formula I and generally one or more solid or liquid formulation adjuvants, are prepared in known manner, e.g. by intimately mixing and/or grinding the active ingredients with the formulation adjuvants, e.g. solvents or solid carriers. Surface-active compounds (surfactants) may additionally be used in the preparation of the formulations. Examples of solvents and solid carriers are given, for example, in WO 97/34485 on page 6.

Depending on the nature of the compound of formula I to be formulated, suitable surface-active compounds are non-ionic, cationic and/or anionic surfactants and surfactant mixtures having good emulsifying, dispersing and wetting properties.

Examples of suitable anionic, non-ionic and cationic surfactants are listed, for example, in WO 97/34485 on pages 7 and 8. The surfactants customarily employed in formulation technology, which are described inter alia in “McCutcheon's Detergents and Emulsifiers Annual” MC Publishing Corp., Ridgewood New Jersey, 1981, Stache, H., “Tensid-Taschenbuch”, Carl Hanser Verlag, Munich/Vienna, 1981 and M. and J. Ash, “Encyclopedia of Surfactants”, Vol I-III, Chemical Publishing Co., New York, 1980-81, are also suitable for the preparation of the herbicidal compositions according to the invention.

The herbicidal formulations generally contain from 0.1 to 99% by weight, especially from 0.1 to 95% by weight, herbicide, from 1 to 99.9% by weight, especially from 5 to 99.8% by weight, of a. solid or liquid formulation adjuvant and from 0 to 25% by weight, especially from 0.1 to 25% by weight, of a surfactant. Whereas commercial products are preferably formulated as concentrates, the end user will normally employ dilute formulations. The compositions may also comprise further ingredients such as stabilisers, e.g. vegetable oils and epoxidised vegetable oils (epoxidised coconut oil, rape oil or soybean oil), anti-foams, e.g. silicone oil, preservatives, viscosity regulators, binders and tackifiers, as well as fertilisers or other active ingredients.

The compounds of formula I are usually applied to the plants or to the locus thereof at rates of application of from 0.001 to 4 kg/ha, especially from 0.005 to 2 kg/ha. The concentration required to achieve the desired action can be determined by experimentation. It will depend on the type of action, the development stage of the crop plant and of the weed, as well as on the application (locus, time, method) and, in dependence on those parameters, can vary over a wide range.

The compounds of formula I are distinguished by herbicidal and growth-inhibiting properties, which make them suitable for use in crops of useful plants, especially in cereals, cotton, soybeans, sugar beet, sugar cane, plantation crops, rape, maize and rice, and for the non-selective control of weeds. Crops will be understood to include also those crops that have been made tolerant to herbicides or classes of herbicides by conventional breeding or genetic engineering methods. The weeds to be controlled may be monocotyledonous as well as dicotyledonous weeds, for example Stellaria, Nasturtium, Agrostis, Digitaria, Avena, Setaria, Sinapis, Lolium, Solanum, Echinochloa, Scirpus, Monochoria, Sagittaria, Bromus, Alopecurus, Sorghum halepense, Rottboellia, Cyperus, Abutilon, Sida, Xanthium, Amaranthus, Chenopodium, Ipomoea, Chrysanthemum, Galium, Viola and Veronica.

The following Examples illustrate the invention further, but do not limit the invention.

### Preparation Examples

### EXAMPLE P1

### Preparation of 3,5-dichloro-2-(prop-2-ynyloxy)-pyridine

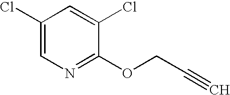

1.25 g (0.029 mol) of NaH (55%) are placed in 30 ml of pentane. After 15 minutes' stirring under nitrogen, the solvent is removed by syringe. 20 ml of absolute THF are then added and 1.65 ml (0.028 mol) of propargyl alcohol are added dropwise over a period of about 5 minutes at a temperature of 0° C. When the addition is complete, the ice-cooling is removed and stirring is continued for a further one hour at a temperature of about 45° C. until the evolution of gas has ceased. 4.8 g (0.025 mol) of 2,3,5-trichloropyridine dissolved in 5 ml of THF are then added dropwise, with stirring, at 45° C. Stirring is then carried out for 6 hours at a temperature of 45° C. and for 18 hours at a temperature of 20° C., until gas chromatography indicates complete conversion. The reaction mixture is then neutralised cautiously with 1N HCl, a small amount of saturated aqueous sodium chloride is added and extraction with ethyl acetate is carried out a total of three times. The combined organic phases are dried over magnesium sulfate. After filtration and removal of the ethyl acetate by evaporation, 5.0 g of 3,5-dichloro-2-(prop-2-ynyloxy)-pyridine are obtained in the form of a pale yellow oil, which corresponds to a quantitative conversion.

### EXAMPLE P2

### Preparation of {3-[3-(3,5-dichloropyridin-2-yloxy)-prop-1-ynyl]-phenyl}-acetonitrile

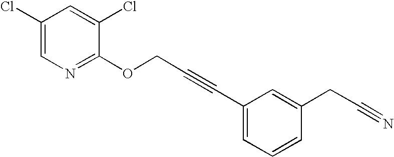

486 mg (2.0 mmol) of 3-iodo-1 -phenylacetonitrile, 17.1 mg (0.09 mmol) of CuI and 126 mg (0.18 mmol) of Pd(PPh₃)₂Cl₂ are dissolved at 20° C. in 10 ml of THF under argon. After 15 minutes' stirring, 0.56 ml (4.0 mmol) of triethylamine is added. A solution of 444 mg (2.2 mmol) of 3,5-dichloro-2-(prop-2-ynyloxy)-pyridine (Example P1) in 3 ml of THF is then added dropwise over a period of 30 minutes. After a further 16 hours' stirring at 20° C., the THF is distilled off, 50 ml of water are added to the residue, and extraction is carried out with a total of about 120 ml of ethyl acetate. After separation of the organic phase, drying over magnesium sulfate and filtration, concentration by evaporation is carried out. The dark residue is purified by chromatography (eluant: ethyl acetate/hexane 1/4). 300 mg of {3-[3-(3,5-dichloropyridin-2-yloxy)-prop-1-ynyl]-phenyl)-acetonitrile are obtained in solid form having a melting point of from 70 to 72° C.

### EXAMPLE P3

### Preparation of 5-bromo-2-(prop-2-ynyloxy)-pyridine

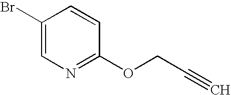

0.54 g (0.011 mol) of NaH (55%) is suspended in 15 ml of absolute THF under nitrogen. 0.67 ml (0.011 mol) of propargyl alcohol is then added dropwise over a period of about 5 minutes at a temperature of 0° C. When the addition is complete, the ice-cooling is removed and stirring is carried out for 30 minutes at a temperature of about 20° C. until the evolution of gas has ceased. 2.0 g (0.011 mol) of 5-bromo-2-fluoropyridine dissolved in 5 ml of THF are then added dropwise at 20-30° C., with stirring and ice-cooling. Stirring is then carried out for a further 2 hours at room temperature until gas chromatography indicates complete conversion. The reaction mixture is then cautiously poured into 40 ml of water and extraction with ethyl acetate is carried out a total of three times. The combined organic phases are dried over sodium sulfate. After filtration and removal of the ethyl acetate by evaporation, 2.1 g of 5-bromo-2-(prop-2-ynyloxy)-pyridine are obtained in the form of beige crystals having a melting point of 58-60° C.

### EXAMPLE P4

### Preparation of 5-chloro-3-fluoro-2-(prop-2-ynyloxy)-pyridine

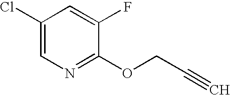

8.0 g (0.167 mol) of NaH (55%) are suspended in 200 ml of absolute THF under nitrogen. 9.9 ml (0.167 mol) of propargyl alcohol dissolved in 10 ml of absolute THF are then added dropwise over a period of about 10 minutes at a temperature of 0° C. When the addition is complete, the ice-cooling is removed and stirring is carried out at room temperature for 45 minutes until the evolution of gas has ceased. 25 g (0.167 mol) of 5-chloro-2,5-difluoropyridine dissolved in 50 ml of THF are then added dropwise at 20-30° C., with stirring and ice-cooling. Stirring is carried out for a further 3 hours at room temperature until gas chromatography indicates complete conversion. The reaction mixture is then cautiously poured into 250 ml of water and extraction with ethyl acetate is carried out a total of three times. After separation of the organic phase, drying over sodium sulfate and filtration, concentration by evaporation is carried out. The yellow residue is purified by chromatography (eluant ethyl acetate/hexane 1/4). 19.1 g of 5-chloro-34fluoro-2-(prop-2-ynyloxy)-pyridine are obtained in the form of a colourless oil.

### EXAMPLE P5

### Preparation of 5-chloro-3-methoxy-2-(prop-2-ynyloxy)-pyridine

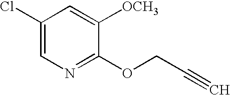

1.0 g (5.4 mmol) of 5-chloro-3-fluoro-2-(prop-2-ynyloxy)-pyridine (Example P4) are placed in 15 ml of methanol under nitrogen. 2.0 ml (10.8 mmol) of a 30% sodium methanolate solution in methanol are then added dropwise over a period of about 5 minutes at room temperature. When the addition is complete, the reaction mixture is heated to reflux and stirred at that temperature for a further 18 hours. The reaction mixture is cooled to room temperature and 30 ml of water are then added cautiously. Extraction with ethyl acetate is then carried out three times. After separation of the organic phase, drying over sodium sulfate and filtration, concentration by evaporation is carried out. The yellowish residue is purified by chromatography (eluant: ethyl acetate/hexane 1/4). 0.65 g of 5-chloro-3-methoxy-2-(prop-2-ynyloxy)-pyridine is obtained in the form of colourless crystals having a melting point of 62-64° C.

### EXAMPLE P6

### Preparation of {3-[3-(5-bromopyridin-2-yloxy)-prop-1-ynyl]-phenyl}-acetonitrile

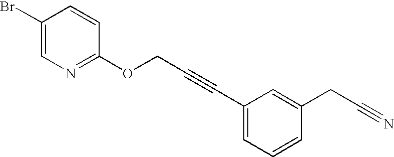

486 mg (2.0 mmol) of 3-iodo-1-phenylacetonitrile, 424 mg (2.0 mmol) of 5-bromo-2-(prop-2-ynyloxy)-pyridine (Example P3) and 80 mg (0.11 mmol) of Pd(PPh₃)₂Cl₂ are dissolved at a temperature of 0° C. in a mixture of 10 ml of chloroform and 4 ml of triethylamine under argon. After 15 minutes' stirring, 36 mg (0.19 mmol) of CuI are added. After a further 18 hours' stirring at a temperature of 0° C., the reaction mixture is filtered through a small amount of silica gel. The filtrate is concentrated by evaporation and the dark residue is purified by chromatography (eluant: ethyl acetate/hexane 1/4). 180 mg of {3-[3-(5-bromo-pyridin-2-yloxy)-prop-1-ynyl]-phenyl}-acetonitrile are obtained in solid form having a melting point of 121-123° C.

### EXAMPLE P7

### Preparation of 5-chloro-2-[3-(4-chlorophenyl)-prop-2-ynyloxy]-3-methoxy-pyridine

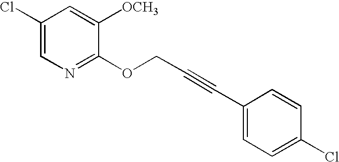

112 mg (0.47 mmol) of 1-iodo-4-chlorobenzene, 94 mg (0.47 mmol) of 5-chloro-3-methoxy-2-(prop-2-ynyloxy)-pyridine (Example P5) and 34 mg (0.047 mmol) of Pd(PPh₃)₂Cl₂ are dissolved at a temperature of −78° C. in a mixture of 5 ml of methanol and 0.335 ml (2.4 mmol) of triethylamine under argon. After 15 minutes' stirring, 27 mg (0.14 mmol) of CuI are added. The reaction mixture is left for 18 hours, with stirring, to warm to room temperature and is then filtered through a small amount of silica gel. The filtrate is concentrated by evaporation and the dark residue is purified by chromatography (eluant:ethyl acetate/hexane 1/4). 37 mg of 5-chloro-2-[3-(4chlorophenyl)-prop-2-ynyloxy]-3-methoxypyridine are obtained in solid form having a melting point of 109-110° C.

TABLE 1
Compounds of formula I

| | |
|---|---|
| | (I) |
| 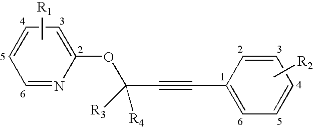 | |

| | | | | | Phys. data |
|---|---|---|---|---|---|
| Comp. | | | | | m.p. |
| No. | R₁ | R₂ | R₃ | R₄ | (° C.) |
| 1.001 | H | 3-Cl | H | H | — |
| 1.002 | H | 3-CH₂CN | H | H | oil |
| 1.003 | H | 3-CH₂CN | CH₃ | H | — |
| 1.004 | H | 3-CN | H | H | — |
| 1.005 | 3-F | 3-CH₂CN | H | H | — |
| 1.006 | 3-Cl | 3-CH₂CN | H | H | — |
| 1.007 | 5-Cl | 3-CH₂CN | H | H | 110-112 |
| 1.008 | 5-Br | 3-CH₂CN | H | H | 121-123 |
| 1.009 | 3-F, 5-F | 3-CH₂CN | H | H | — |
| 1.010 | 3-Cl, 5-Cl | 3-CH₂CN | H | H | 70-72 |
| 1.011 | 3-F, 5-Cl | 3-CH₂CN | H | H | 64-66 |
| 1.012 | 3-F, 5-Cl | 3-CH₂CN | CH₃ | H | — |
| 1.013 | 3-F, 5-Cl | 3-CH₂CN | CH₃ | CH₃ | — |
| 1.014 | 3-OCH₃, 5-CF₃ | 3-CH₂CN | H | H | — |
| 1.015 | 3-OCH₃, 5-F | 3-CH₂CN | H | H | — |
| 1.016 | 3-OCH₃, 5-Cl | 3-CH₂CN | H | H | 93-97 |
| 1.017 | 3-OCH₃, 5-Br | 3-CH₂CN | H | H | — |
| 1.018 | 3-OCH₃, 5-F | 3-CH₂CN | CH₃ | H | — |
| 1.019 | 3-OCH₃, 5-Cl | 3-CH₂CN | CH₃ | H | — |
| 1.020 | 3-OCH₃, 5-F | 3-CH(CH₃)CN | H | H | — |
| 1.021 | 3-OCH₃, 5-Cl | 3-CH(CH₃)CN | H | H | — |
| 1.022 | 3-OCH₃, 5-CH₃ | 3-CH₂CN | H | H | — |
| 1.023 | 3-OCH₃, 5-CH₃ | 3-CH(CH₃)CN | H | H | — |
| 1.024 | 3-OCHF₂, 6-CH₃ | 3-CH₂CN | H | H | 32-34 |
| 1.025 | 3-OCHF₂, 5-Cl | 3-CH₂CN | H | H | — |
| 1.026 | 3-OCHF₂, 5-F | 3-CH₂CN | H | H | — |
| 1.027 | 3-OCH₃, 5-CH═NOCH₃ | 3-CH₂CN | H | H | — |
| 1.028 | 3-OCH₃, 5-Cl | 4-Cl | H | H | 109-110 |
| 1.029 | 3-OCH₃, 5-Cl | 3-Cl | H | H | 79-82 |
| 1.030 | 3-OCH₃, 5-Cl | 3-Br | H | H | 82-85 |
| 1.031 | 3-CH₃, 5-NHCOCH₃ | 3-CH₂CN | H | H | — |
| 1.032 | 3-CH₃, 5-Cl | 3-CH₂CN | H | H | — |
| 1.033 | 3-CH₃, 5-F | 3-CH₂CN | H | H | — |
| 1.034 | 3-OCH₃, 5-CH═NOCH₃ | 3-CH(CH₃)CN | H | H | — |
| 1.035 | 3-OCH₃, 5-CH═NOCH₃ | 3-CH₂CN | CH₃ | H | — |
| 1.036 | 3-Cl, 5-Cl, 6-OCH₃ | 3-CH₂CN | H | H | 91-93 |
| 1.037 | 3-Cl, 5-F, 6-OCH₃ | 3-CH₂CN | H | H | — |
| 1.038 | 3-F, 5-CF₃ | 3-CH₂CN | H | H | — |
| 1.039 | 3-Cl, 5-CF₃ | 3-CH₂CN | H | H | oil |
| 1.040 | 3-Br, 5-CF₃ | 3-CH₂CN | H | H | — |
| 1.041 | 3-F, 5-CF₃ | 3-CH(CH₃)CN | H | H | — |
| 1.042 | 3-Cl, 5-CF₃ | 3-CH(CH₃)CN | H | H | — |
| 1.043 | 3-F, 5-CF₃ | 3-CH₂CN | CH₃ | H | — |
| 1.044 | 3-Cl, 5-CF₃ | 3-CH₂CN | CH₃ | H | — |
| 1.045 | 3-SCH₃, 5-Cl | 3-CH₂CN | H | H | — |
| 1.046 | 3-SCH₃, 5-F | 3-CH₂CN | H | H | — |
| 1.047 | 3-SCH₃ | 3-CH₂CN | H | H | — |
| 1.048 | 3-S-iso-C₃H₇ | 3-CH₂CN | H | H | oil |
| 1.049 | 3-OCH₃, 5-CN | 3-CH₂CN | H | H | — |
| 1.050 | 3-OCH₃, 5-CN | 3-CH₂CN | CH₃ | H | — |
| 1.051 | 3-OCH₃, 5-CN | 4-Br | H | H | — |
| 1.052 | 3-OCH₃, 5-F | 3-CH₂CN | CH₃ | CH₃ | — |
| 1.053 | 3-OCH₃, 5-Cl | 3-CH₂CN | CH₃ | CH₃ | — |
| 1.054 | H | 3-CH₂CN | CH₃ | CH₃ | — |
| 1.055 | 3-OCH₃, 5-CH═NOCH₃ | 3-Cl | H | H | — |
| 1.056 | 3-OCH₃, 5-CH═NOCH₃ | 4-Cl | H | H | — |
| 1.057 | 3-Cl, 5-Cl, 6-Cl | 3-CH₂CN | H | H | — |
| 1.058 | 3-Cl, 5-Cl, 6-F | 3-CH₂CN | H | H | — |
| 1.059 | 3-Cl, 5-Cl, 6-F | 3-CH₂CN | CH₃ | H | — |
| 1.060 | 3-OCH₃, 5-Cl | 3-CN | H | H | — |
| 1.061 | 3-OCH₃, 5-F | 3-CN | H | H | — |
| 1.062 | 3-OCH₃, 5-CF₃ | 3-CN | H | H | — |
| 1.063 | 3-OCH₃, 5-Cl | 4-Br | H | H | — |
| 1.064 | 3-OCH₃, 5-F | 4-Br | H | H | — |
| 1.065 | 3-F, 5-Cl | 4-Br | H | H | — |
| 1.066 | 3-F, 5-Cl | 3-CH(CH₃)CN | H | H | — |
| 1.067 | 3-F, 5-Cl | 3-CH(CH₃)CN | CH₃ | H | — |
| 1.068 | 3-F, 5-Cl | 3-CH(CH₃)CN | CH₃ | CH₃ | — |
| 1.069 | 3-OCH₃, 5-Cl | 3-CH(CH₃)CN | CH₃ | H | — |
| 1.070 | 3-OCH₃, 5-Cl | 3-CH(CH₃)CN | CH₃ | CH₃ | — |
| 1.071 | 3-OCH₃, 5-F | 3-CH(CH₃)CN | CH₃ | H | — |
| 1.072 | 3-OCH₃, 5-F | 3-CH(CH₃)CN | CH₃ | CH₃ | — |
| 1.073 | 3-OCH₃, 5-Cl | 3-C(CH₃)₂CN | H | H | — |
| 1.074 | 3-OCH₃, 5-F | 3-C(CH₃)₂CN | H | H | — |
| 1.075 | 3-CF₃ | 3-CH₂CN | H | H | — |
| 1.076 | 3-CF₃, 5-Cl | 3-CH₂CN | H | H | — |
| 1.077 | 3-CF₃, 5-F | 3-CH₂CN | H | H | — |
| 1.078 | 3-OCH₃, 5-Cl | 3-C(S)NH₂ | H | H | — |
| 1.079 | 3-OCH₃, 5-F | 3-C(S)NH₂ | H | H | — |
| 1.080 | 3-F, 5-Cl | 3-C(S)NH₂ | H | H | — |
| 1.081 | 3-OCH₃, 5-Cl | 4-NO₂ | H | H | resin |
| 1.082 | 3-OCH₃, 5-Cl | 2-CONH₂ | H | H | resin |
| 1.083 | 3-OCH₃, 5-Cl | 4-CO₂CH₃ | H | H | resin |
| 1.084 | 3-OCH₃, 5-Cl | 2-F, 3-F, 4-F, 5-F, 6-F | H | H | solid |
| 1.085 | 3-OCH₃, 5-Cl | 3-CH₃, 4-CH₃ | H | H | resin |
| 1.086 | 3-OCH₃, 5-Cl | 3-CH₃, 5-CH₃ | H | H | resin |
| 1.087 | 3-OCH₃, 5-Cl | 2-OCF₃, 4-Br | H | H | resin |
| 1.088 | 3-OCH₃, 5-Cl | 4-F | H | H | resin |
| 1.089 | 3-OCH₃, 5-Cl | 2-F, 4-F | H | H | resin |
| 1.090 | 3-OCH₃, 5-Cl | 3-CH₃, 4-F | H | H | resin |
| 1.091 | 3-OCH₃, 5-Cl | 2-F, 4-Cl | H | H | resin |
| 1.092 | 3-OCH₃, 5-Cl | 4-CF₃ | H | H | 89-90 |
| 1.093 | 3-OCH₃, 5-Cl | 4-CO₂C₂H₅ | H | H | 126-127 |
| 1.094 | 3-OCH₃, 5-Cl | H | H | H | solid |
| 1.095 | 3-OCH₃, 5-Cl | 2-Cl, 4-Cl | H | H | resin |
| 1.096 | 3-OCH₃, 5-Cl | 2-Cl, 5-CF₃ | H | H | resin |
| 1.097 | 3-OCH₃, 5-Cl | 2-F, 4-Br | H | H | resin |
| 1.098 | 3-OCH₃, 5-Cl | 3-CF₃ | H | H | resin |
| 1.099 | 3-OCH₃, 5-Cl | 2-Cl | H | H | resin |
| 1.100 | 3-OCH₃, 5-Cl | 3-F, 4-CH₃ | H | H | resin |
| 1.101 | 3-OCH₃, 5-Cl | 3-Cl, 4-F | H | H | resin |
| 1.102 | 3-OCH₃, 5-Cl | 2-CH₃, 3-Cl | H | H | resin |
| 1.103 | 3-OCH₃, 5-Cl | 2-Cl, 4-CF₃ | H | H | resin |
| 1.104 | 3-OCH₃, 5-Cl | 2-Cl, 4-Br | H | H | resin |
| 1.105 | 3-OCH₃, 5-Cl | 4-CO₂C₂H₅ | H | H | resin |
| 1.106 | 3-OCH₃, 5-F | 3-CF₃, 5-CF₃ | H | H | resin |
| 1.107 | 3-OCH₃, 5-F | 2-Cl, 4-Cl, 5-Cl | H | H | resin |
| 1.108 | 3-OCH₃, 5-F | 2-CH₃, 4-Cl | H | H | resin |
| 1.109 | 3-OCH₃, 5-F | 4-CO₂CH₃ | H | H | resin |
| 1.110 | 3-OCH₃, 5-F | 2-F, 4-Cl, 5-CO₂CH₃ | H | H | resin |
| 1.111 | 3-OCH₃, 5-F | 5-CO₂C₂H₅ | H | H | resin |
| 1.112 | 3-OCH₃, 5-F | 2-CN, 3-F | H | H | resin |
| 1.113 | 3-OCH₃, 5-F | 4-NO₂ | H | H | resin |
| 1.114 | 3-OCH₃, 5-F | 3-Cl, 4-CH₃ | H | H | resin |
| 1.115 | 3-OCH₃, 5-F | 3-Cl, 6-OCH₃ | H | H | resin |
| 1.116 | 3-OCH₃, 5-F | 4-CF₃ | H | H | resin |
| 1.117 | 3-OCH₃, 5-F | 2-Cl, 5-Cl | H | H | resin |
| 1.118 | 3-CN, 4-CH₃, 6-CH₃ | 3-CH₂CN | H | H | amorphous |
| 1.119 | 3-SO₂NH₂ | 3-CH₂CN | H | H | solid |
| 1.120 | 3-OCH₃, 5-Cl | 2-Cl, 4-F | H | H | 80-82 |
| 1.121 | 3-CN, 5-CN | 3-CH₂CN | H | H | solid |
| 1.122 | 3-OCH₃, 5-F | 3-OCH₃ | H | H | 102-104 |
| 1.123 | 3-OCH₃, 5-Cl | 3-F | H | H | 79-82 |
| 1.124 | 3-OCH₃, 5-F | 3-Cl | H | H | solid |
| 1.125 | 3-OCH₃, 5-Cl | 3-NO₂ | H | H | 137-139 |
| 1.126 | 3-OCH₃, 5-Cl | 2-Cl, 3-Cl | H | H | 104-106 |
| 1.127 | 3-F, 5-F | 2-Cl, 5-Cl | H | H | resin |
| 1.128 | 3-F, 5-F | 3-Cl, 4-CH₃ | H | H | resin |
| 1.129 | 3-F, 5-F | 2-Cl, 4-Cl, 5-Cl | H | H | resin |
| 1.130 | 3-F, 5-F | 4-CH₃ | H | H | resin |
| 1.131 | 3-F, 5-F | 3-OCF₃ | H | H | resin |
| 1.132 | 3-F, 5-F | 3-OCH₂C₆H₅ | H | H | resin |
| 1.133 | 3-F, 5-F | 3-CN | H | H | resin |
| 1.134 | 3-F, 5-F | 3-CF₃, 5-CF₃ | H | H | resin |
| 1.135 | 3-F, 5-F | 3-F, 4-F | H | H | resin |
| 1.136 | 6-CF₂Cl | 3-CH₂CN | H | H | oil |
| 1.137 | 3-CN, 6-CH₃ | 3-CH₂CN | H | H | amorphous |
| 1.138 | 6-CF₃ | 3-CH₂CN | H | H | oil |
| 1.139 | 3-F, 5-F | 2-F, 4-Cl, 5-CO₂CH₃ | H | H | resin |
| 1.140 | 3-F, 5-F | 3-CO₂C₂H₅ | H | H | resin |
| 1.141 | 3-OCH₃, 5-Cl | 3-F, 4-F | H | H | resin |
| 1.142 | 3-F, 5-F | 3-F | H | H | resin |
| 1.143 | 3-F, 5-F | 3-Cl, 6-OCH₃ | H | H | resin |
| 1.144 | 3-F, 5-F | 2-CN, 3-F | H | H | resin |
| 1.145 | 3-F, 5-F | 4-CF₃ | H | H | resin |
| 1.146 | 3-CO₂C₂H₅, 6-CF₃ | 3-CH₂CN | H | H | oil |
| 1.147 | 5-CF₃ | 3-CH₂CN | H | H | oil |
| 1.148 | 3-NO₂, 5-Cl | 3-Cl | H | H | 98-99 |
| 1.149 | 3-F, 5-F | 4-NO₂ | H | H | resin |
| 1.150 | 3-OCH₃, 5-F | 3-Br | H | H | — |
| 1.151 | 3-CF₃, 5-F | 3-Br | H | H | — |
| 1.152 | 3-F, 5-Cl | 3-Br | H | H | — |
| 1.153 | 3-Cl, 5-Cl | 3-Br | H | H | — |
| 1.154 | 3-F,5-F | 3-Br | H | H | — |
| 1.155 | 3-OCH₃, 5-Br | 3-Br | H | H | — |
| 1.156 | 3-F, 5-Cl | 3-OSO₂CH₃ | H | H | — |
| 1.157 | 3-F, 5-F | 3-OSO₂CH₃ | H | H | — |
| 1.158 | 3-Cl, 5-Cl | 3-OSO₂CH₃ | H | H | — |
| 1.159 | 3-OCH₃, 5-Cl | 3-OSO₂CH₃ | H | H | — |
| 1.160 | 3-OCH₃, 5-Br | 3-OSO₂CH₃ | H | H | — |
| 1.161 | 3-OCH₃, 5-F | 3-OSO₂CH₃ | H | H | — |
| 1.162 | 3-OCH₃, 5-CH═NOCH₃ | 3-OSO₂CH₃ | H | H | — |
| 1.163 | 3-OCH₃, 5-Br | 3-Cl | H | H | — |
| 1.164 | 3-OCH₃, 5-CF₃ | 3-Cl | H | H | — |
| 1.165 | 3-F, 5-Cl | 3-Cl | H | H | — |
| 1.166 | 3-F, 5-F | 3-Cl | H | H | — |
| 1.167 | 3-OCH₃, 5-F | 3-OCH₃ | H | H | — |
| 1.168 | 3-F, 5-Cl | 3-OCH₃ | H | H | — |
| 1.169 | 3-Cl, 5-Cl | 3-OCH₃ | H | H | — |
| 1.170 | 3-OCH₃, 5-Br | 3-OCH₃ | H | H | — |
| 1.171 | 3-F, 5-F | 3-OCH₃ | H | H | — |
| 1.172 | 3-OCH₃, 5-Cl | 3-OCH₃ | H | H | — |
| 1.173 | 3-F, 5-F | 3-CH(CH₃)CN | H | H | — |
| 1.174 | 3-OCH₃, 5-CF₃ | 3-CH(CH₃)CN | H | H | — |
| 1.175 | 3-OCH₃, 5-CN | 3-Br | H | H | — |
| 1.176 | 3-OCH₃, 5-CN | 3-OSO₂CH₃ | H | H | — |
| 1.177 | 3-OCH₃, 5-CN | 3-Cl | H | H | — |
| 1.178 | 3-OCH₃, 5-CN | 3-OCH₃ | H | H | — |
| 1.179 | 3-OCH₃, 5-CN | 3-I | H | H | — |
| 1.180 | 3-F, 5-Cl | 3-I | H | H | — |
| 1.181 | 3-Cl, 5-Cl | 3-I | H | H | — |
| 1.182 | 3-OCH₃, 5-F | 3-I | H | H | — |
| 1.183 | 3-OCH₃, 5-Cl | 3-I | H | H | — |
| 1.184 | 3-OCH₃, 5-Br | 3-I | H | H | — |
| 1.185 | 3-CF₃, 5-F | 3-I | H | H | — |
| 1.186 | 3-OCH₃, 5-CF₃ | 3-I | H | H | — |
| 1.187 | 3-F, 5-F | 3-C(S)NH₂ | H | H | — |
| 1.188 | 3-OCH₃, 5-CF₃ | 3-C(S)NH₂ | H | H | — |
| 1.189 | 3-Cl, 5-Cl | 3-C(S)NH₂ | H | H | — |
| 1.190 | 3-OCH₃, 5-Br | 3-C(S)NH₂ | H | H | — |
| 1.191 | 3-OCH₃, 5-CN | 3-CH₂—CCH | H | H | — |
| 1.192 | 3-F, 5-Cl | 3-CH₂—CCH | H | H | — |
| 1.193 | 3-Cl, 5-Cl | 3-CH₂—CCH | H | H | — |
| 1.194 | 3-OCH₃, 5-F | 3-CH₂—CCH | H | H | — |
| 1.195 | 3-OCH₃, 5-Cl | 3-CH₂—CCH | H | H | — |
| 1.196 | 3-OCH₃, 5-Br | 3-CH₂—CCH | H | H | — |
| 1.197 | 3-CF₃, 5-F | 3-CH₂—CCH | H | H | — |
| 1.198 | 3-OCH₃, 5-CF₃ | 3-CH₂—CCH | H | H | — |
| 1.199 | 3-OCH₃, 5-CH═NOCH₃ | 3-CH₂—CCH | H | H | — |
| 1.200 | 3-OCH₃, 5-CN | 3-CH₂—CH═CH₂ | H | H | — |
| 1.201 | 3-F, 5-Cl | 3-CH₂—CH═CH₂ | H | H | — |
| 1.202 | 3-Cl, 5-Cl | 3-CH₂—CH═CH₂ | H | H | — |
| 1.203 | 3-OCH₃, 5-F | 3-CH₂—CH═CH₂ | H | H | — |
| 1.204 | 3-OCH₃, 5-Cl | 3-CH₂—CH═CH₂ | H | H | — |
| 1.205 | 3-OCH₃, 5-Br | 3-CH₂—CH═CH₂ | H | H | — |
| 1.206 | 3-CF₃, 5-F | 3-CH₂—CH═CH₂ | H | H | — |
| 1.207 | 3-OCH₃, 5-CF₃ | 3-CH₂—CH═CH₂ | H | H | — |
| 1.208 | 3-OCH₃, 5-CH₃ | 3-CH₂—CH═CH₂ | H | H | — |
| 1.209 | 3-OCH₃, 5-CH═NOCH₃ | 3-CH₂—CH═CH₂ | H | H | — |
| 1.210 | 3-Cl, 5-Cl | 4-Br | H | H | — |
| 1.211 | 3-OCH₃, 5-Br | 4-Br | H | H | — |
| 1.212 | 3-CF₃, 5-F | 4-Br | H | H | — |
| 1.213 | 3-OCH₃, 5-CF₃ | 4-Br | H | H | — |
| 1.214 | 3-OCH₃, 5-CH₃ | 4-Br | H | H | — |
| 1.215 | 3-OCH₃, 5-CH═NOCH₃ | 4-Br | H | H | — |
| 1.216 | 3-OCH₃, 5-CN | 4-Cl | H | H | — |
| 1.217 | 3-F, 5-Cl | 4-Cl | H | H | — |
| 1.218 | 3-Cl, 5-Cl | 4-Cl | H | H | — |
| 1.219 | 3-OCH₃, 5-F | 4-Cl | H | H | — |
| 1.220 | 3-OCH₃, 5-Br | 4-Cl | H | H | — |
| 1.221 | 3-CF₃, 5-F | 4-Cl | H | H | — |
| 1.222 | 3-OCH₃, 5-CF₃ | 4-Cl | H | H | — |
| 1.223 | 3-OCH₃, 5-CH₃ | 4-Cl | H | H | — |
| 1.224 | 3-Cl, 5-Cl | 3-Cl | H | H | — |
| 1.225 | 3-CF₃, 5-F | 3-Cl | H | H | — |
| 1.226 | 3-OCH₃, 5-CH₃ | 3-Cl | H | H | — |
| 1.227 | 3-OCH₃, 5-CN | 4-CH₂CN | H | H | — |
| 1.228 | 3-F, 5-Cl | 4-CH₂CN | H | H | — |
| 1.229 | 3-Cl, 5-Cl | 4-CH₂CN | H | H | — |
| 1.230 | 3-OCH₃, 5-F | 4-CH₂CN | H | H | — |
| 1.231 | 3-OCH₃, 5-Cl | 4-CH₂CN | H | H | — |
| 1.232 | 3-OCH₃, 5-Br | 4-CH₂CN | H | H | — |
| 1.233 | 3-CF₃, 5-F | 4-CH₂CN | H | H | — |
| 1.234 | 3-OCH₃, 5-CF₃ | 4-CH₂CN | H | H | — |
| 1.235 | 3-OCH₃, 5-CH₃ | 4-CH₂CN | H | H | — |
| 1.236 | 3-OCH₃, 5-CH═NOCH₃ | 4-CH₂CN | H | H | — |
| 1.237 | 3-OCH₃, 5-CN | 3-CHO | H | H | — |
| 1.238 | 3-F, 5-Cl | 3-CHO | H | H | — |
| 1.239 | 3-Cl, 5-Cl | 3-CHO | H | H | — |
| 1.240 | 3-OCH₃, 5-F | 3-CHO | H | H | — |
| 1.241 | 3-OCH₃, 5-Cl | 3-CHO | H | H | — |
| 1.242 | 3-OCH₃, 5-Br | 3-CHO | H | H | — |
| 1.243 | 3-CF₃, 5-F | 3-CHO | H | H | — |
| 1.244 | 3-OCH₃, 5-CF₃ | 3-CHO | H | H | — |
| 1.245 | 3-OCH₃, 5-CH₃ | 3-CHO | H | H | — |
| 1.246 | 3-OCH₃, 5-CH═NOCH₃ | 3-CHO | H | H | — |
| 1.247 | 3-OCH₃, 5-CN | 3-CH₂OH | H | H | — |
| 1.248 | 3-F, 5-Cl | 3-CH₂OH | H | H | — |
| 1.249 | 3-Cl, 5-Cl | 3-CH₂OH | H | H | — |
| 1.250 | 3-OCH₃, 5-F | 3-CH₂OH | H | H | — |
| 1.251 | 3-OCH₃, 5-Cl | 3-CH₂OH | H | H | — |
| 1.252 | 3-OCH₃, 5-Br | 3-CH₂OH | H | H | — |
| 1.253 | 3-CF₃, 5-F | 3-CH₂OH | H | H | — |
| 1.254 | 3-OCH₃, 5-CF₃ | 3-CH₂OH | H | H | — |
| 1.255 | 3-OCH₃, 5-CH₃ | 3-CH₂OH | H | H | — |
| 1.256 | 3-OCH₃, 5-CH═NOCH₃ | 3-CH₂OH | H | H | — |
| 1.257 | 3-NO₂, 6-Cl | 3-CH₂CN | H | H | — |
| 1.258 | 3-NO₂, 6-CF₃ | 3-CH₂CN | H | H | — |
| 1.259 | 3-NO₂, 6-OCH₃ | 3-CH₂CN | H | H | — |
| 1.260 | 3-NO₂, 5-OCH₃ | 3-CH₂CN | H | H | — |
| 1.261 | 3-F, 5-OCH₃ | 3-CH₂CN | H | H | — |
| 1.262 | 3-Cl, 5-OCH₃ | 3-CH₂CN | H | H | — |
| 1.263 | 3-OCH₃, 5-OCH₃ | 3-CH₂CN | H | H | — |
| 1.264 | 3-F, 5-OC₆H₅ | 3-CH₂CN | H | H | — |
| 1.265 | 3-Cl, 5-OC₆H₅ | 3-CH₂CN | H | H | — |
| 1.266 | 3-F, 5-OCH₂C₆H₅ | 3-CH₂CN | H | H | — |
| 1.267 | 3-Cl, 5-OCH₂C₆H₅ | 3-CH₂CN | H | H | — |
| 1.268 | 3-OCH₃, 5-OCH₂C₆H₅ | 3-CH₂CN | H | H | — |
| 1.269 | 3-OCH₃, 5-CN | 3-CH(OCH₃)CN | H | H | — |
| 1.270 | 3-F, 5-Cl | 3-CH(OCH₃)CN | H | H | — |
| 1.271 | 3-Cl, 5-Cl | 3-CH(OCH₃)CN | H | H | — |
| 1.272 | 3-OCH₃, 5-F | 3-CH(OCH₃)CN | H | H | — |
| 1.273 | 3-OCH₃, 5-Cl | 3-CH(OCH₃)CN | H | H | — |
| 1.274 | 3-OCH₃, 5-Br | 3-CH(OCH₃)CN | H | H | — |
| 1.275 | 3-CF₃, 5-F | 3-CH(OCH₃)CN | H | H | — |
| 1.276 | 3-OCH₃, 5-CF₃ | 3-CH(OCH₃)CN | H | H | — |
| 1.277 | 3-OCH₃, 5-CH₃ | 3-CH(OCH₃)CN | H | H | — |
| 1.278 | 3-OCH₃, 5-CH═NOCH₃ | 3-CH(OCH₃)CN | H | H | — |
| 1.279 | 3-OCH₃, 5-CN | 3-CH(OCH₃)₂ | H | H | — |
| 1.280 | 3-F, 5-Cl | 3-CH(OCH₃)₂ | H | H | — |
| 1.281 | 3-Cl, 5-Cl | 3-CH(OCH₃)₂ | H | H | — |
| 1.282 | 3-OCH₃, 5-F | 3-CH(OCH₃)₂ | H | H | — |
| 1.283 | 3-OCH₃, 5-Cl | 3-CH(OCH₃)₂ | H | H | — |
| 1.284 | 3-OCH₃, 5-Br | 3-CH(OCH₃)₂ | H | H | — |
| 1.285 | 3-CF₃, 5-F | 3-CH(OCH₃)₂ | H | H | — |
| 1.286 | 3-OCH₃, 5-CF₃ | 3-CH(OCH₃)₂ | H | H | — |
| 1.287 | 3-OCH₃, 5-CH₃ | 3-CH(OCH₃)₂ | H | H | — |
| 1.288 | 3-OCH₃, 5-CH═NOCH₃ | 3-CH(OCH₃)₂ | H | H | — |
| 1.289 | 3-OCH₃, 5-CN | 3-CH₂Br | H | H | — |
| 1.290 | 3-F, 5-Cl | 3-CH₂Br | H | H | — |
| 1.291 | 3-Cl, 5-Cl | 3-CH₂Br | H | H | — |
| 1.292 | 3-OCH₃, 5-F | 3-CH₂Br | H | H | — |
| 1.293 | 3-OCH₃, 5-Cl | 3-CH₂Br | H | H | — |
| 1.294 | 3-OCH₃, 5-Br | 3-CH₂Br | H | H | — |
| 1.295 | 3-CF₃, 5-F | 3-CH₂Br | H | H | — |
| 1.296 | 3-OCH₃, 5-CF₃ | 3-CH₂Br | H | H | — |
| 1.297 | 3-OCH₃, 5-CH₃ | 3-CH₂Br | H | H | — |
| 1.298 | 3-OCH₃, 5-CH═NOCH₃ | 3-CH₂Br | H | H | — |
| 1.299 | 3-OCH₃, 5-CN | 3-CH₂CONH₂ | H | H | — |
| 1.300 | 3-F, 5-Cl | 3-CH₂CONH₂ | H | H | — |
| 1.301 | 3-Cl, 5-Cl | 3-CH₂CONH₂ | H | H | — |
| 1.302 | 3-OCH₃, 5-F | 3-CH₂CONH₂ | H | H | — |
| 1.303 | 3-OCH₃, 5-Cl | 3-CH₂CONH₂ | H | H | — |
| 1.304 | 3-OCH₃, 5-Br | 3-CH₂CONH₂ | H | H | — |
| 1.305 | 3-CF₃, 5-F | 3-CH₂CONH₂ | H | H | — |
| 1.306 | 3-OCH₃, 5-CF₃ | 3-CH₂CONH₂ | H | H | — |
| 1.307 | 3-OCH₃, 5-CH₃ | 3-CH₂CONH₂ | H | H | — |
| 1.308 | 3-OCH₃, 5-CH═NOCH₃ | 3-CH₂CONH₂ | H | H | — |
| 1.309 | 3-F, 5-F | 3-CH₂CN | CH₃ | CH₃ | — |
| 1.310 | 3-Cl, 5-Cl | 3-CH₂CN | CH₃ | CH₃ | — |
| 1.311 | 3-OCH₃, 5-Br | 3-CH₂CN | CH₃ | CH₃ | — |
| 1.312 | 3-OCH₃, 5-CH₃ | 3-CH₂CN | CH₃ | CH₃ | — |
| 1.313 | 3-F, 5-F | 3-CH₂CN | (CH₂)₂ | | — |
| 1.314 | 3-F, 5-Cl | 3-CH₂CN | (CH₂)₂ | | — |
| 1.315 | 3-OCH₃, 5-F | 3-CH₂CN | (CH₂)₂ | | — |
| 1.316 | 3-OCH₃, 5-Cl | 3-CH₂CN | (CH₂)₂ | | — |
| 1.317 | 3-F, 5-F | 3-CH₂CN | (CH₂)₄ | | — |
| 1.318 | 3-F, 5-Cl | 3-CH₂CN | (CH₂)₄ | | — |
| 1.319 | 3-OCH₃, 5-F | 3-CH₂CN | (CH₂)₄ | | — |
| 1.320 | 3-OCH₃, 5-Cl | 3-CH₂CN | (CH₂)₄ | | — |
| 1.321 | 3-F, 5-F | 3-CH(CH₃)CN | (CH₂)₂ | | — |
| 1.322 | 3-F, 5-Cl | 3-CH(CH₃)CN | (CH₂)₂ | | — |
| 1.323 | 3-OCH₃, 5-F | 3-CH(CH₃)CN | (CH₂)₂ | | — |
| 1.324 | 3-OCH₃, 5-CN | 3-CH═NOCH₃ | H | H | — |
| 1.325 | 3-F, 5-Cl | 3-CH═NOCH₃ | H | H | — |
| 1.326 | 3-Cl, 5-Cl | 3-CH═NOCH₃ | H | H | — |
| 1.327 | 3-OCH₃, 5-F | 3-CH═NOCH₃ | H | H | — |
| 1.328 | 3-OCH₃, 5-Cl | 3-CH═NOCH₃ | H | H | — |
| 1.329 | 3-OCH₃, 5-Br | 3-CH═NOCH₃ | H | H | — |
| 1.330 | 3-CF₃, 5-F | 3-CH═NOCH₃ | H | H | — |
| 1.331 | 3-OCH₃, 5-CF₃ | 3-CH═NOCH₃ | H | H | — |
| 1.332 | 3-OCH₃, 5-CH₃ | 3-CH═NOCH₃ | H | H | — |
| 1.333 | 3-OCH₃, 5-CH═NOCH₃ | 3-CH═NOCH₃ | H | H | — |

TABLE 2
Compounds of formula I

| | |
|---|---|
| | (I) |
| 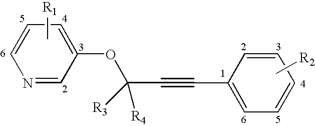 | |

| Comp. | | | | | Phys. data. |
|---|---|---|---|---|---|
| No. | R₁ | R₂ | R₃ | R₄ | m.p. (° C.) |
| 2.001 | 2-Cl, 6-F | 3-Cl | H | H | — |
| 2.002 | 2-Cl, 6-F | 4-Cl | H | H | — |
| 2.003 | 2-Cl, 6-F | 3-CH₂CN | H | H | — |
| 2.004 | 2-Cl, 6-F | 3-OSO₂CH₃ | H | H | — |
| 2.005 | 2-Cl, 6-Cl | 3-Cl | H | H | — |
| 2.006 | 2-Cl, 6-Cl | 4-Cl | H | H | — |
| 2.007 | 2-Cl, 6-Cl | 3-CH₂CN | H | H | 85-86 |
| 2.008 | 2-Cl, 6-Cl | 3-OSO₂CH₃ | H | H | — |
| 2.009 | 2-Cl, 6-Br | 3-Cl | H | H | — |
| 2.010 | 2-Cl, 6-Br | 4-Cl | H | H | — |
| 2.011 | 2-Cl, 6-Br | 3-CH₂CN | H | H | resin |
| 2.012 | 2-Cl, 6-Br | 3-OSO₂CH₃ | H | H | — |
| 2.013 | 2-F, 6-CH₃ | 3-Cl | H | H | — |
| 2.014 | 2-Cl, 6-CH₃ | 3-Cl | H | H | — |
| 2.015 | 2-F, 6-CH₃ | 3-CH₂CN | H | H | — |
| 2.016 | 2-Cl, 6-CH₃ | 3-CH₂CN | H | H | — |
| 2.017 | 2-F, 6-OCH₃ | 3-CH₂CN | H | H | — |
| 2.018 | 2-Cl, 6-OCH₃ | 3-CH₂CN | H | H | — |
| 2.019 | 2-F | 3-Cl | H | H | — |
| 2.020 | 2-F | 3-CH₂CN | H | H | — |
| 2.021 | 2-Cl | 3-Cl | H | H | — |
| 2.022 | 2-Cl | 3-CH₂CN | H | H | oil |
| 2.023 | 2-Br | 3-Cl | H | H | — |
| 2.024 | 2-Br | 3-CH₂CN | H | H | resin |
| 2.025 | 2-CF₃ | 3-CH₂CN | H | H | — |
| 2.026 | 2-CH₂OH | 3-CH₂CN | H | H | solid |
| 2.027 | 2-NO₂, 6-OCH₃ | 3-Cl | H | H | — |
| 2.028 | 2-NO₂, 6-OCH₃ | 4-Cl | H | H | — |
| 2.029 | 2-NO₂, 6-OCH₃ | 3-CH₂CN | H | H | — |
| 2.030 | 2-NO₂, 6-OCH₃ | 3-OSO₂CH₃ | H | H | — |
| 2.031 | 2-NO₂, 6-CH₃ | 4-Cl | H | H | — |
| 2.032 | 2-NO₂, 6-CH₃ | 3-CH₂CN | H | H | oil |
| 2.033 | 2-F, 6-CF₃ | 3-Cl | H | H | — |
| 2.034 | 2-F, 6-CF₃ | 4-Cl | H | H | — |
| 2.035 | 2-F, 6-CF₃ | 3-CH₂CN | H | H | — |
| 2.036 | 2-F, 6-CF₃ | 3-OSO₂CH₃ | H | H | — |
| 2.037 | 2-Cl, 6-CF₃ | 3-Cl | H | H | — |
| 2.038 | 2-Cl, 6-CF₃ | 4-Cl | H | H | — |
| 2.039 | 2-Cl, 6-CF₃ | 3-CH₂CN | H | H | — |
| 2.040 | 2-Cl, 6-CF₃ | 3-OSO₂CH₃ | H | H | — |
| 2.041 | 2-CF₃, 6-CH₃ | 3-CH₂CN | H | H | — |
| 2.042 | 2-CF₃, 6-OCH₃ | 3-CH₂CN | H | H | — |
| 2.043 | 2-CF₃, 6-CF₃ | 3-CH₂CN | H | H | — |
| 2.044 | 2-CO₂C₂H₅, 5-Cl | 3-CH₂CN | H | H | solid |
| 2.045 | 2-Cl, 6-F | 3-Cl | CH₃ | H | — |
| 2.046 | 2-Cl, 6-F | 4-Cl | CH₃ | H | — |
| 2.047 | 2-Cl, 6-F | 3-CH₂CN | CH₃ | H | — |
| 2.048 | 2-Cl, 6-F | 3-OSO₂CH₃ | CH₃ | H | — |
| 2.049 | 2-Cl, 6-Cl | 3-Cl | CH₃ | H | — |
| 2.050 | 2-Cl, 6-Cl | 4-Cl | CH₃ | H | — |
| 2.051 | 2-Cl, 6-Cl | 3-CH₂CN | CH₃ | H | — |
| 2.052 | 2-Cl, 6-Cl | 3-OSO₂CH₃ | CH₃ | H | — |
| 2.053 | 2-Cl, 6-Br | 3-Cl | CH₃ | H | — |
| 2.054 | 2-Cl, 6-Br | 4-Cl | CH₃ | H | — |
| 2.055 | 2-Cl, 6-Br | 3-CH₂CN | CH₃ | H | — |
| 2.056 | 2-Cl, 6-Br | 3-OSO₂CH₃ | CH₃ | H | — |
| 2.057 | 2-F, 6-CH₃ | 3-Cl | CH₃ | H | — |
| 2.058 | 2-Cl, 6-CH₃ | 3-Cl | CH₃ | H | — |
| 2.059 | 2-F, 6-CH₃ | 3-CH₂CN | CH₃ | H | — |
| 2.060 | 2-Cl, 6-CH₃ | 3-CH₂CN | CH₃ | H | — |
| 2.061 | 2-F, 6-OCH₃ | 3-CH₂CN | CH₃ | H | — |
| 2.062 | 2-Cl, 6-OCH₃ | 3-CH₂CN | CH₃ | H | — |
| 2.063 | 2-F | 3-Cl | CH₃ | H | — |
| 2.064 | 2-F | 3-CH₂CN | CH₃ | H | — |
| 2.065 | 2-Cl | 3-Cl | CH₃ | H | — |
| 2.066 | 2-Cl | 3-CH₂CN | CH₃ | H | — |
| 2.067 | 2-Br | 3-Cl | CH₃ | H | — |
| 2.068 | 2-Br | 3-CH₂CN | CH₃ | H | — |
| 2.069 | 2-CF₃ | 3-CH₂CN | CH₃ | H | — |
| 2.070 | 2-CH₂OH | 3-CH₂CN | CH₃ | H | — |
| 2.071 | 2-NO₂, 6-OCH₃ | 3-Cl | CH₃ | H | — |
| 2.072 | 2-NO₂, 6-OCH₃ | 4-Cl | CH₃ | H | — |
| 2.073 | 2-NO₂, 6-OCH₃ | 3-CH₂CN | CH₃ | H | — |
| 2.074 | 2-NO₂, 6-OCH₃ | 3-OSO₂CH₃ | CH₃ | H | — |
| 2.075 | 2-NO₂, 6-CH₃ | 4-Cl | CH₃ | H | — |
| 2.076 | 2-NO₂, 6-CH₃ | 3-CH₂CN | CH₃ | H | — |
| 2.077 | 2-F, 6-CF₃ | 3-Cl | CH₃ | H | — |
| 2.078 | 2-F, 6-CF₃ | 4-Cl | CH₃ | H | — |
| 2.079 | 2-F, 6-CF₃ | 3-CH₂CN | CH₃ | H | — |
| 2.080 | 2-F, 6-CF₃ | 3-OSO₂CH₃ | CH₃ | H | — |
| 2.081 | 2-Cl, 6-CF₃ | 3-Cl | CH₃ | H | — |
| 2.082 | 2-Cl, 6-CF₃ | 4-Cl | CH₃ | H | — |
| 2.083 | 2-Cl, 6-CF₃ | 3-CH₂CN | CH₃ | H | — |
| 2.084 | 2-Cl, 6-CF₃ | 3-OSO₂CH₃ | CH₃ | H | — |
| 2.085 | 2-CF₃, 6-CH₃ | 3-CH₂CN | CH₃ | H | — |
| 2.086 | 2-CF₃, 6-OCH₃ | 3-CH₂CN | CH₃ | H | — |
| 2.087 | 2-CF₃, 6-CF₃ | 3-CH₂CN | CH₃ | H | — |
| 2.088 | 2-CO₂C₂H₅, 5-Cl | 3-CH₂CN | CH₃ | H | — |
| 2.089 | 4-F, 6-F | 3-CH₂CN | H | H | — |
| 2.090 | 4-Cl, 6-F | 3-CH₂CN | H | H | — |
| 2.091 | 4-Cl, 6-Cl | 3-CH₂CN | H | H | — |
| 2.092 | 4-Cl, 6-Br | 3-CH₂CN | H | H | — |
| 2.093 | 4-F, 6-CH₃ | 3-CH₂CN | H | — | — |
| 2.094 | 4-Cl, 6-CH₃ | 3-CH₂CN | H | — | — |
| 2.095 | 4-F, 6-OCH₃ | 3-CH₂CN | H | — | — |
| 2.096 | 4-Cl, 6-OCH₃ | 3-CH₂CN | H | — | — |
| 2.097 | 4-NO₂, 6-OCH₃ | 3-CH₂CN | H | — | — |
| 2.098 | 4-NO₂, 6-CH₃ | 3-CH₂CN | H | — | — |
| 2.099 | 4-F, 6-CF₃ | 3-CH₂CN | H | — | — |
| 2.100 | 4-Cl, 6-CF₃ | 3-CH₂CN | H | — | — |
| 2.101 | 4-CF₃, 6-CH₃ | 3-CH₂CN | H | — | — |
| 2.102 | 4-CF₃, 6-OCH₃ | 3-CH₂CN | H | — | — |
| 2.103 | 4-CF₃, 6-CF₃ | 3-CH₂CN | H | — | — |
| 2.104 | 2-Cl, 6-piperidyl | 3-CH₂CN | H | — | resin |

TABLE 3
Compounds of formula I

| | |
|---|---|
| | (I) |
| 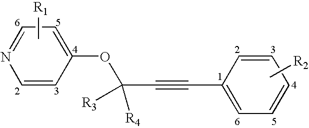 | |

| Comp. | | | | | Phys. data |
|---|---|---|---|---|---|
| No. | R₁ | R₂ | R₃ | R₄ | m.p. (° C.) |
| 3.001 | 2-F | 3-Cl | H | H | — |
| 3.002 | 2-Cl | 3-Cl | H | H | — |
| 3.003 | 2-Br | 3-Cl | H | H | — |
| 3.004 | 2-F | 3-CH₂CN | H | H | — |
| 3.005 | 2-Cl | 3-CH₂CN | H | H | — |
| 3.006 | 2-Br | 3-CH₂CN | H | H | — |
| 3.007 | 3-F | 3-Cl | H | H | — |
| 3.008 | 3-Cl | 3-Cl | H | H | — |
| 3.009 | 3-Br | 3-Cl | H | H | — |
| 3.010 | 3-F | 3-CH₂CN | H | H | — |
| 3.011 | 3-Cl | 3-CH₂CN | H | H | — |
| 3.012 | 3-Br | 3-CH₂CN | H | H | — |
| 3.013 | 2-CF₃ | 3-CH₂CN | H | H | oil |
| 3.014 | 3-CF₃ | 3-CH₂CN | H | H | — |
| 3.015 | 3-CF₂Cl | 3-CH₂CN | H | H | — |
| 3.016 | 3-F | 3-CH(CH₃)CN | H | H | — |
| 3.017 | 3-Cl | 3-CH(CH₃)CN | H | H | — |
| 3.018 | 3-Br | 3-CH(CH₃)CN | H | H | — |
| 3.019 | 3-F | CH(OCH₃)CN | H | H | — |
| 3.020 | 3-Cl | CH(OCH₃)CN | H | H | — |
| 3.021 | 3-Br | CH(OCH₃)CN | H | H | — |
| 3.022 | 3-F | 3-OSO₂CH₃ | H | H | — |
| 3.023 | 3-Cl | 3-OSO₂CH₃ | H | H | — |
| 3.024 | 3-Br | 3-OSO₂CH₃ | H | H | — |
| 3.025 | 3-F | CH(OCH₃)₂ | H | H | — |
| 3.026 | 3-Cl | CH(OCH₃)₂ | H | H | — |
| 3.027 | 3-Br | CH(OCH₃)₂ | H | H | — |
| 3.028 | 2-F, 5-F | 3-CH₂CN | H | H | — |
| 3.029 | 2-Cl, 5-F | 3-CH₂CN | H | H | — |
| 3.030 | 3-F | 4-Cl | H | H | — |
| 3.031 | 3-Cl | 4-Cl | H | H | — |
| 3.032 | 3-Br | 4-Cl | H | H | — |
| 3.033 | 2-CH₃, 5-F | 3-CH₂CN | H | H | — |
| 3.034 | 2-CH₃, 5-Cl | 3-CH₂CN | H | H | — |
| 3.035 | 2-CH₃, 5-Br | 3-CH₂CN | H | H | — |
| 3.036 | 2-F | 3-Cl | CH₃ | H | — |
| 3.037 | 2-Cl | 3-Cl | CH₃ | H | — |
| 3.038 | 2-Br | 3-Cl | CH₃ | H | — |
| 3.039 | 2-F | 3-CH₂CN | CH₃ | H | — |
| 3.040 | 2-Cl | 3-CH₂CN | CH₃ | H | — |
| 3.041 | 2-Br | 3-CH₂CN | CH₃ | H | — |
| 3.042 | 3-F | 3-Cl | CH₃ | H | — |
| 3.043 | 3-Cl | 3-Cl | CH₃ | H | — |
| 3.044 | 3-Br | 3-Cl | CH₃ | H | — |
| 3.045 | 3-F | 3-CH₂CN | CH₃ | H | — |
| 3.046 | 3-Cl | 3-CH₂CN | CH₃ | H | — |
| 3.047 | 3-Br | 3-CH₂CN | CH₃ | H | — |
| 3.048 | 2-CF₃ | 3-CH₂CN | CH₃ | H | — |
| 3.049 | 3-CF₃ | 3-CH₂CN | CH₃ | H | — |
| 3.050 | 3-CF₂Cl | 3-CH₂CN | CH₃ | H | — |
| 3.051 | 3-F | 3-CH(CH₃)CN | CH₃ | H | — |
| 3.052 | 3-Cl | 3-CH(CH₃)CN | CH₃ | H | — |
| 3.053 | 3-Br | 3-CH(CH₃)CN | CH₃ | H | — |
| 3.054 | 3-F | CH(OCH₃)CN | CH₃ | H | — |
| 3.055 | 3-Cl | CH(OCH₃)CN | CH₃ | H | — |
| 3.056 | 3-Br | CH(OCH₃)CN | CH₃ | H | — |
| 3.057 | 3-F | OSO₂CH₃ | CH₃ | H | — |
| 3.058 | 3-Cl | OSO₂CH₃ | CH₃ | H | — |
| 3.059 | 3-Br | OSO₂CH₃ | CH₃ | H | — |
| 3.060 | 3-F | CH(OCH₃)₂ | CH₃ | H | — |
| 3.061 | 3-Cl | CH(OCH₃)₂ | CH₃ | H | — |
| 3.062 | 3-Br | CH(OCH₃)₂ | CH₃ | H | — |
| 3.063 | 2-F, 5-F | 3-CH₂CN | CH₃ | H | — |
| 3.064 | 2-Cl, 5-F | 3-CH₂CN | CH₃ | H | — |
| 3.065 | 3-F | 4-Cl | CH₃ | H | — |
| 3.066 | 3-Cl | 4-Cl | CH₃ | H | — |
| 3.067 | 3-Br | 4-Cl | CH₃ | H | — |
| 3.068 | 2-CH₃, 5-F | 3-CH₂CN | CH₃ | H | — |
| 3.069 | 2-CH₃, 5-Cl | 3-CH₂CN | CH₃ | H | — |
| 3.070 | 2-CH₃, 5-Br | 3-CH₂CN | CH₃ | H | — |

### Biological Examples

### EXAMPLE B1

### Herbicidal Action Before Emergence of the Plants (Pre-Emergence Action)

Monocotyledonous and dicotyledonous test plants are sown in standard soil in pots. Immediately after sowing, an aqueous suspension of the test compounds (prepared from a wettable powder (Example F3, b) according to WO 97/34485) or an emulsion of the test compounds (prepared from an emulsifiable concentrate (Example F1, c) according to WO 97/34485) is applied by spraying at an optimum rate of application (500 litres of water/ha). The test plants are then cultivated in a greenhouse under optimum conditions.

After a test duration of 4 weeks, the test is evaluated in accordance with a scale of nine ratings (1=total damage, 9=no action). Ratings of from 1 to 4 (especially from 1 to 3) indicate good to very good herbicidal action.

Test plants: Setaria, Panicum, Digitaria, Amaranthus, Stellaria, Veronica.

TABLE B1
Rate of application: 1000 g a.i./ha

| Comp. | Seta- | | | Amaran- | | |
|---|---|---|---|---|---|---|
| No. | ria | Panicum | Digitaria | thus | Stellaria | Veronica |
| 1.011 | 6 | 1 | 1 | 1 | 1 | 1 |
| 1.008 | 7 | 1 | 1 | 1 | 4 | 1 |
| 1.016 | 4 | 2 | 1 | 1 | 1 | 1 |
| 1.007 | 5 | 1 | 1 | 1 | 1 | 7 |
| 1.009 | — | — | 1 | 2 | 3 | — |
| 1.015 | 7 | 2 | 2 | 1 | 1 | 4 |
| 1.017 | 5 | 2 | 2 | 1 | 1 | 2 |
| 1.022 | — | 2 | — | 1 | 2 | 1 |
| 1.024 | 1 | — | — | 1 | 1 | 1 |
| 1.025 | 1 | 1 | 2 | 1 | 1 | 1 |
| 1.047 | 1 | 2 | 1 | 1 | 1 | 4 |
| 1.094 | — | — | — | 3 | 1 | 1 |

The same results are obtained when the compounds of formula I are formulated according to the other Examples analogously to WO 97/34485.

### EXAMPLE B2

### Post-Emergence Herbicidal Action

Monocotyledonous and dicotyledonous test plants are sown in standard soil in pots, and at the 2- to 3-leaf stage are sprayed with an aqueous suspension of the test compounds (prepared from a wettable powder (Example F3, b) according to WO 97/34485) or with an emulsion of the test compounds (prepared from an emulsifiable concentrate (Example F1, c) according to WO 97/34485) at an optimum rate of application of 500 litres of water/ha). The test plants are then grown on in the greenhouse under optimum conditions.

After a test duration of 2 to 3 weeks, the test is evaluated in accordance with a scale of nine ratings (1=total damage, 9=no action). Ratings of from 1 to 4 (especially from 1 to 3) indicate good to very good herbicidal action.

Test plants: Panicum, Euphorbia, Sida, Amaranthus, Chenopodium, Stellaria, Veronica.

TABLE B2
Rate of application: 1000 g a.i/ha

| Comp. | Pani- | Euphor- | | Amaran- | Cheno- | Stell- | |
|---|---|---|---|---|---|---|---|
| No. | cum | bia | Sida | thus | podium | aria | Veronica |
| 1.011 | 2 | 2 | 2 | 2 | 1 | 2 | 2 |
| 1.008 | 7 | 2 | 3 | 1 | 1 | 4 | 2 |
| 1.016 | 2 | 1 | 2 | 1 | 1 | 1 | 1 |
| 1.007 | 2 | 2 | 4 | 1 | 1 | 6 | 1 |
| 1.009 | 2 | 1 | 2 | 1 | 1 | 2 | 2 |
| 1.015 | 1 | 1 | 3 | 1 | 1 | 1 | 2 |
| 1.017 | 4 | 1 | 2 | 2 | 1 | 1 | 2 |
| 1.022 | 6 | 2 | 3 | 3 | 3 | 4 | 3 |
| 1.024 | — | 2 | 2 | 1 | 1 | 1 | 3 |
| 1.025 | — | 2 | 3 | 2 | 7 | 4 | 4 |
| 1.047 | 4 | 1 | 3 | 2 | 1 | 1 | 3 |
| 1.094 | 2 | 1 | 2 | 1 | 3 | 2 | 3 |

In the above Tables B1 and B2, “-” indicates that there are no data for the corresponding indication.

Th same results are obtained when the compounds of formula I are formulated according to the other Examples analogously to WO 97/34485.

What is claimed is:

1. A compound of formula I 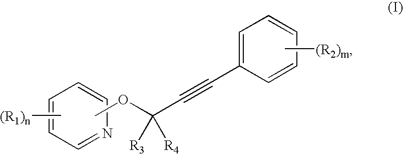
wherein
n is 0, 1, 2, 3 or 4;
each R₁ independently of any other(s) is halogen, —CN, —SCN, —SF₅, —NO₂, —NR₅R₆, —CO₂R₇, —CONR₈R₉, —C(R₁₀)═NOR₁₁, —COR₁₂, —OR₁₃, —SR₁₄, —SOR₁₅, —SO₂R₁₆, —OSO₂R₁₇, C₁-C₈alkyl, C₂-C₈alkenyl, C₂-C₈alkynyl or C₃-C₆cycloalkyl; or C₁-C₈alkyl, C₂-C₈alkenyl or C₂-C₈alkynyl substituted by one or more halogen, —CN, —NO₂, —NR₁₈R₁₉, —CO₂R₂₀, —CONR₂₁R₂₂, —COR₂₃, —C(R₂₄)═NOR₂₅, —C(S)NR₂₆R₂₇, —C(C₁-C₄alkylthio)=NR₂₈, —OR₂₉, —SR₃₀, —SOR₃₁, —SO₂R₃₂ or C₃-C₆cycloalkyl substituents; or
each R₁ is C₃-C₆cycloalkyl substituted by one or more halogen, —CN, —NO₂, —NR₁₈R₁₉, —CO₂R₂₀, —CONR₂₁R₂₂, —COR₂₃, —C(R₂₄)═NOR₂₅, —C(S)NR₂₆R₂₇, —C(C₁-C₄alkylthio)=NR₂₈, —SR₃₀, —SOR₃₁, —SO₂R₃₂ or C₃-C₆cycloalkyl substituents; or
each R₁ independently of any other(s) is phenyl, which may itself be substituted by one or more halogen, C₁-C₄alkyl, C₁-C₄haloalkyl, C₁-C₄alkoxy, —CN, —NO₂, C₁-C₄alkylthio, C₁-C₄-alkylsulfinyl or C₁-C₄alkylsulfonyl substituents; or
two adjacent R₁ together form a C₁-C₇alkylene bridge, which may be interrupted by 1 or 2 non-adjacent oxygen atoms and may be substituted by C₁-C₆alkyl, the total number of ring atoms being at least 5 and a maximum of 9; or two adjacent RI together form a C₂-C₇-alkenylene bridge, which may be interrupted by 1 or 2 non-adjacent oxygen atoms and may be substituted by C₁-C₆alkyl, the total number of ring atoms being at least 5 and a maximum of 9;
R₃ and R₄ are each independently of the other hydrogen, halogen, —CN, C₁-C₄alkyl or C₁-C₄-alkoxy; or
R₃ and R₄ together denote C₂-C₅alkylene;
R₅ is hydrogen or C₁-C₈alkyl;
R₆ is hydrogen, C₁-C₈alkyl, C₃-C₈alkenyl, C₃-C₈alkynyl, phenyl or benzyl; it being possible for phenyl and benzyl themselves to be substituted by one or more halogen, C₁-C₄alkyl, C₁-C₄-haloalkyl, C₁-C₄alkoxy, —CN, —NO₂, C₁-C₄alkylthio, C₁-C₄alkylsulfinyl or C₁-C₄alkylsulfonyl substituents; or
R₅ and R₆ together denote a C₂-C₅alkylene chain, which may be interrupted by an oxygen or sulfur atom;
R₇ is hydrogen, C₁-C₈alkyl, C₃-C₈alkenyl or C₃-C₈alkynyl, or C₁-C₈alkyl, C₃-C₈alkenyl or C₃-C₈alkynyl substituted by one or more halogen, C₁-C₄alkoxy or phenyl substituents, it being possible for phenyl itself to be substituted by one or more halogen, C₁-C₄alkyl, C₁-C₄-haloalkyl, C₁-C₄alkoxy, —CN, —NO₂, C₁-C₄alkylthio, C₁-C₄alkylsulfinyl or C₁-C₄alkylsulfonyl substituents;
R₈ is hydrogen or C₁-C₈alkyl;
R₉ is hydrogen or C₁-C₈alkyl, or C₁-C₈alkyl substituted by one or more COOH, C₁-C₈-alkoxycarbonyl or N substituents, or
R₉ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or
R₈ and R₉ together denote C₂-C₅alkylene;
R₁₀ is hydrogen, C₁-C₄alkyl, C₁-C₄haloalkyl or C₃-C₆cycloalkyl;
R₁₁ is hydrogen, C₁-C₈alkyl, C₃-C₈alkenyl, C₃-C₈alkynyl, C₁-C₄haloalkyl or C₃-C₆haloalkenyl;
R₁₂ is hydrogen, C₁-C₄alkyl, C₁-C₄haloalkyl or C₃-C₆cycloalkyl;
R₁₃ is hydrogen, C₁-C₈alkyl, C₃-C₈alkenyl or C₃-C₈alkynyl; or
R₁₃ is phenyl or phenyl-C₁-C₆alkyl, it being possible for the phenyl ring itself to be substituted by one or more halogen, C₁-C₄alkyl, C₁-C₄haloalkyl, C₁-C₄alkoxy, —CN, —NO₂ or —S(O)₂C₁-C₈alkyl substituents, or
R₁₃ is C₁-C₈alkyl substituted by one or more halogen, —CN or C₁-C₄alkoxy substituents;
R₁₄ is hydrogen, C₁-C₈alkyl, C₃-C₈alkenyl or C₃-C₈alkynyl, or C₁-C₈alkyl substituted by one or more halogen, —CN or C₁-C₄alkoxy substituents;
R₁₅, R₁₆ and R₁₇ are each independently of the others C₁-C₈alkyl, C₃-C₈alkenyl or C₃-C₈-alkynyl, or C₁-C₈alkyl substituted by one or more halogen, —CN or C₁-C₄alkoxy substituents;
R₁₈ is hydrogen or C₁-C₈alkyl;
R₁₉ is hydrogen, C₁-C₈alkyl, C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkyl-sulfonyl substituents; or
R₁₈ and R₁₉ together denote a C₂-C₅alkylene chain, which may be interrupted by an oxygen or sulfur atom;
R₂₀ is hydrogen, C₁-C₈alkyl, C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkyl-sulfonyl substituents;
R₂₁ is hydrogen or C₁-C₈alkyl;
R₂₂ is hydrogen or C₁-C₈alkyl, or C₁-C₈alkyl substituted by one or more COOH, C₁-C₈-alkoxycarbonyl or —CN substituents, or
R₂₂ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or
R₂₁ and R₂₂together denote C₂-C₅alkylene;
R₂₃ is hydrogen, C₁-C₄alkyl, C₁-C₄haloalkyl or C₃-C₆cycloalkyl;
R₂₄ is hydrogen, C₁-C₄alkyl, C₁-C₄haloalkyl or C₃-C₆cycloalkyl;
R₂₅ is hydrogen, C₁-C₈alkyl, C₃-C₈alkenyl, C₃-C₈alkynyl, C₁-C₄haloalkyl or C₃-C₆haloalkenyl;
R₂₆ is hydrogen or C₁-C₈alkyl;
R₂₇ is hydrogen or C₁-C₈alkyl, or C₁-C₈alkyl substituted by one or more COOH, C₁-C₈-alkoxycarbonyl or —CN substituents, or
R₂₇ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or
R₂₆ and R₂₇ together denote C₂-C₅alkylene;
R₂₈ is hydrogen or C₁-C₈alkyl;
R₂₉ and R₃₀ are each independently of the other hydrogen, C₁-C₈alkyl, C₃-C₈alkenyl or C₃-C₈alkynyl, or C₁-C₈alkyl substituted by one or more halogen, —CN or C₁-C₄alkoxy substituents;
R₃₁ and R₃₂ are each independently of the other C₁-C₈alkyl, C₃-C₈alkenyl or C₃-C₈alkynyl, or C₁-C₈alkyl substituted by one or more halogen, —CN or C₁-C₄alkoxy substituents;
m is 0, 1, 2, 3, 4 or 5;
each R₂ independently of any other(s) is halogen, —CN, —SCN, —SF₅, —NO₂, —NR₃₆R₃₇, —CO₂R₃₈, —CONR₃₉R₄₀, —C(R₄₁)═NOR₄₂, —COR₄₃, —OR₄₄, —SR₄₅, —SOR₄₆, —SO₂R₄₇, OSO₂R₄₈—N([CO]ₚR₄₉)COR₅₀, —N(OR₅₁)COR₅₂, —N(R₅₃)CO₂R₅₄ or —N-phthalimide;
R₃₆ is hydrogen or C₁-C₈alkyl; and
R₃₇ is hydrogen, C₁-C₈alkyl, C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkyl-sulfonyl substituents; or
R₃₆ and R₃₇ together denote a C₂-C₅alkylene chain, which may be interrupted by an oxygen or sulfur atom;
R₃₈ is hydrogen, C₁-C₈alkyl, C₃-C₈alkenyl or C₃-C₈alkynyl, or C₁-C₈alkyl, C₃-C₈alkenyl or C₃-C₈alkynyl substituted by one or more halogen, C₁-C₄alkoxy or phenyl substituents, it being possible for phenyl itself to be substituted by one or more halogen, C₁-C₄alkyl, C₁-C₄-haloalkyl, C₁-C₄alkoxy, —CN, —NO₂, C₁-C₄alkylthio, C₁-C₄alkylsulfinyl or C₁-C₄alkylsulfonyl substituents;
R₃₉ is hydrogen or C₁-C₈alkyl;
R₄₀ is hydrogen or C₁-C₈alkyl, or C₁-C₈alkyl substituted by one or more —COOH, C₁-C₈-alkoxycarbonyl or —CN substituents, or
R₄₀ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or
R₃₉ and R₄₀ together denote C₃-C₅alkylene;
R₄₁ is hydrogen, C₁-C₄alkyl, C₁-C₄haloalkyl or C₃-C₆cycloalkyl;
R₄₂ is hydrogen, C₁-C₈alkyl, C₃-C₈alkenyl, C₃-C₈alkynyl, C₁-C₄haloalkyl or C₃-C₆haloalkenyl;
R₄₃ is hydrogen, C₁-C₄alkyl, C₁-C₄haloalkyl or C₃-C₆cycloalkyl;
R₄₄ is hydrogen, C₁-C₈alkyl, C₃-C₈alkenyl or C₃-C₈alkynyl; or
R₄₄ is phenyl or phenyl-C₁-C₆alkyl, it being possible for the phenyl ring itself to be substituted by one or more halogen, C₁-C₄alkyl, C₁-C₄haloalkyl, C₁-C₄alkoxy, —CN, —NO₂ or —S(O)₂C₁-C₈alkyl substituents, or
R₄₄ is C₁-C₈alkyl substituted by one or more halogen, —CN or C₁-C₄alkoxy substituents;
R₄₅ is hydrogen, C₁-C₈alkyl, C₃-C₈alkenyl or C₃-C₈alkynyl, or C₁-C₈alkyl substituted by one or more halogen, —CN or C₁-C₄alkoxy substituents;
R₄₆, R₄₇ and R₄₈ are each independently of the others C₁-C₈alkyl, C₃-C₈alkenyl or C₃-C₈-alkynyl, or C₁-C₈alkyl substituted by one or more halogen, —CN or C₁-C₄alkoxy substituents;
p is 0 or 1;
R₄₉, R₅₀, R₅₁, R₅₂, R₅₃ and R₅₄ are each independently of the others hydrogen, C₁-C₈alkyl, or phenyl, which may itself be substituted by one or more halogen, C₁-C₈alkyl, C₁-C₄haloalkyl, C₁-C₄alkoxy, —CN, —NO₂, C₁-C₈alkylthio, C₁-C₈alkylsulfinyl or C₁-C₈alkylsulfonyl substituents; or
each R₂ independently of any other(s) is C₁-C₈alkyl, or C₁-C₈alkyl mono- or poly-substituted by halogen, —CN, —NO₂, —NR₅₅R₅₆, —CO₂R₅₇, —CONR₅₈R₅₉, —COR₆₀, —C(R₆₁)═NOR₆₂, —C(S)NR₆₃R₆₄, —C(C₁-C₄alkylthio)=NR₆₅, —OR₆₆, —SR₆₇, —SOR₆₈, —SO₂R₆₉, —O(SO₂)R₇₀, —N(R₇₁)CO₂R₇₂, —N(R₇₃)COR₇₄ or by C₃-C₆cycloalkyl; or
each R₂ independently of any other(s) is C₂-C₈alkenyl, or C₂-C₈alkenyl mono- or poly-substituted by —CN, —NO₂, —CO₂R₇₅, —CONR₇₆R₇₇, —COR₇₈, —C(R₇₉)═NOR₈₀, —C(S)NR₈₁R₈₂, —C(C₁-C₄alkylthio)=NR₈₃ or by C₃-C₆cycloalkyl; or
each R₂ independently of any other(s) is C₂-C₈alkynyl, or C₂-C₈alkynyl mono- or poly-substituted by halogen, —CN, —CO₂R₈₄, —CONR₈₅R₈₆, —COR₈₇, —C(R₈₈)═NOR₈₉, —C(S)NR₉₀R₉₁, —C(C₁-C₄alkylthio)=NR₉₂ or by C₃-C₆cycloalkyl; or
each R₂ independently of any other(s) is C₃-C₆cycloalkyl, or C₃-C₆cycloalkyl mono- or poly-substituted by halogen, —CN, —CO₂R₉₃, —CONR₉₄R₉₅, —COR₉₆, —C(R₉₇)═NOR₉₈, —C(S)NR₉₉R₁₀₀ or by —C(C₁-C₄alkylthio)=NR₁₀₁; or
two adjacent R₂ together form a C₁-C₇alkylene bridge, which may be interrupted by 1 or 2 non-adjacent oxygen atoms and may be substituted by C₁-C₆alkyl, the total number of ring atoms being at least 5 and a maximum of 9; or two adjacent R₂ together form a C₂-C₇-alkenylene bridge, which may be interrupted by 1 or 2 non-adjacent oxygen atoms and may be substituted by C₁-C₆alkyl, the total number of ring atoms being at least 5 and a maximum of 9;
R₅₅ is hydrogen or C₁-C₈alkyl;
R₅₆ is hydrogen, C₁-C₅₈alkyl, C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₅₅ and R₅₆ together denote a C₂-C₅alkylene chain, which may be interrupted by an oxygen or sulfur atom;
R₅₇ is hydrogen, C₁-C₈alkyl, C₃-C₈alkenyl or C₃-C₈alkynyl, or C₁-C₈alkyl, C₃-C₈alkenyl or C₃-C₈alkynyl substituted by one or more halogen, C₁-C₄alkoxy or phenyl substituents, it being possible for phenyl itself to be substituted by one or more halogen, C₁-C₄alkyl, C₁-C₄-haloalkyl, C₁-C₄alkoxy, —CN, —NO₂, C₁-C₄alkylthio, C₁-C₄alkylsulfinyl or C₁-C₄alkylsulfonyl substituents;
R₅₈ is hydrogen or C₁-C₈alkyl;
R₅₉ is hydrogen or C₁-C₈alkyl, or C₁-C₈alkyl substituted by one or more —COOH, C₁-C₈-alkoxycarbonyl or —CN substituents; or
R₅₉ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or
R₅₈ and R₅₉ together denote C₂-C₆alkylene;
R₆₀ is hydrogen, C₁-C₄alkyl, C₁-C₄haloalkyl or C₃-C₆cycloalkyl;
R₆₁ is hydrogen, C₁-C₄alkyl, C₁-C₄haloalkyl or C₃-C₆cycloalkyl;
R₆₂ is hydrogen, C₁-C₈alkyl, C₃-C₈alkenyl, C₃-C₈alkynyl, C₁-C₄haloalkyl or C₃-C₆haloalkenyl; and
R₆₃ is hydrogen or C₁-C₈alkyl;
R₆₄ is hydrogen or C₁-C₈alkyl, or C₁-C₈alkyl substituted by one or more —COOH, C₁-C₈-alkoxycarbonyl or —CN substituents; or
R₆₄ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or
R₆₃ and R₆₄together denote C₂-C₅alkylene;
R₆₅ is hydrogen or C₁-C₈alkyl;
R₆₆ and R₆₇ are each independently of the other hydrogen, C₁-C₈alkyl, C₃-C₈alkenyl, C₃-C₈-alkynyl, or C₁-C₈alkyl substituted by one or more halogen, —CN or C₁-C₄alkoxy substituents;
R₆₈, R₆₉ and R₇₀ are each independently of the others C₁-C₈alkyl, C₃-C₈alkenyl or C₃-C₈-alkynyl, or C₁-C₈alkyl substituted by one or more halogen, —CN or C₁-C₄alkoxy substituents;
R₇₁ and R₇₃ are each independently of the other hydrogen, C₁-C₈alkyl or C₁-C₈alkoxy;
R₇₂ is C₁-C₈alkyl;
R₇₄ is hydrogen or C₁-C₈alkyl;
R₇₅ is hydrogen, C₁-C₈alkyl, C₃-C₈alkenyl or C₃-C₈alkynyl, each of which may be mono- or poly-substituted by one or more halogen, C₁-C₄alkoxy or phenyl substituents, it being possible for phenyl itself to be substituted by one or more halogen, C₁-C₄alkyl, C₁-C₄-haloalkyl, C₁-C₄alkoxy, —CN, —NO₂, C₁-C₄alkylthio, C₁-C₄alkylsulfinyl or C₁-C₄alkylsulfonyl substituents;
R₇₆ is hydrogen or C₁-C₈alkyl;
R₇₇ is hydrogen or C₁-C₈alkyl, or C₁-C₈alkyl substituted by one or more —COOH, C₁-C₈-alkoxycarbonyl or N substituents; or
R₇₇ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or
R₇₆and R₇₇together denote C₂-C₅alkylene;
R₇₈ and R₇₉ are each independently of the other hydrogen, C₁-C₄alkyl, C₁-C₄haloalkyl or C₃-C₆cycloalkyl; R₈₀ is hydrogen, C₁-C₈alkyl, C₃-C₈alkenyl, C₃-C₈alkynyl, C₁-C₄haloalkyl or C₃-C₆haloalkenyl;
R₈₁ is hydrogen or C₁-C₈alkyl;
R₈₂ is hydrogen or C₁-C₈alkyl, or C₁-C₈alkyl substituted by one or more —COOH, C₁-C₈-alkoxycarbonyl or —CN substituents; or
R₈₂ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or
R₈₁ and R₈₂ together denote C₂-C₅alkylene;
R₈₃ is hydrogen or C₁-C₈alkyl;
R₈₄ is hydrogen, C₁-C₈alkyl, C₃-C₈alkenyl or C₃-C₈alkynyl, each of which may be mono- or poly-substituted by one or more halogen, C₁-C₄alkoxy or phenyl substituents, it being possible for phenyl itself to be substituted by one or more halogen, C₁-C₄alkyl, C₁-C₄-haloalkyl, C₁-C₄alkoxy, —CN, —NO₂, C₁-C₄alkylthio, C₁-C₄alkylsulfinyl or C₁-C₄alkylsulfonyl substituents;
R₈₅ is hydrogen or C₁-C₈alkyl;
R₈₆ is hydrogen or C₁-C₈alkyl, or C₁-C₈alkyl substituted by one or more —COOH, C₁-C₈-alkoxycarbonyl or —CN substituents; or
R₈₆ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or
R₈₅ and R₈₆ together denote C₂-C₅alkylene;
R₈₇ is hydrogen, C₁-C₄alkyl, C₁-C₄haloalkyl or C₃-C₆cycloalkyl;
R₈₈ is hydrogen, C₁-C₄alkyl, C₁-C₄haloalkyl or C₃-C₆cycloalkyl;
R₈₉ is hydrogen, C₁-C₈alkyl, C₃-C₈alkenyl, C₃-C₈alkynyl, C₁-C₄haloalkyl or C₃-C₆haloalkenyl;
R₉₀ is hydrogen or C₁-C₈alkyl;
R₉₁ is hydrogen or C₁-C₈alkyl, or C₁-C₈alkyl substituted by one or more —COOH, C₁-C₈-alkoxycarbonyl or —CN substituents; or
R₉₁ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or
R₉₀ and R₉₁ together denote C₂-C₅alkylene;
R₉₂ is hydrogen or C₁-C₈alkyl;
R₉₃ is hydrogen, C₁-C₈alkyl, C₃-C₈alkenyl or C₃-C₈alkynyl, each of which may be mono- or poly-substituted by one or more halogen, C₁-C₄alkoxy or phenyl substituents, it being possible for phenyl itself to be substituted by one or more halogen, C₁-C₄alkyl, C₁-C₄-haloalkyl, C₁-C₄alkoxy, —CN, —NO₂, C₁-C₄alkylthio, C_{1-C}₄alkylsulfinyl or C₁-C₄alkylsulfonyl substituents;
R₉₄ is hydrogen or C₁-C₈alkyl;
R₉₅ is hydrogen or C₁-C₈alkyl, or C₁-C₈alkyl substituted by one or more —COOH, C₁-C₈-alkoxycarbonyl or —CN substituents; or
R₉₅ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or
R₉₄ and R₉₅ together denote C₂-C₅alkylene;
R₉₆ is hydrogen, C₁-C₄alkyl, C₁-C₄haloalkyl or C₃-C₆cycloalkyl;
R₉₇ is hydrogen, C₁-C₄alkyl, C₁-C₄haloalkyl or C₃-C₆cycloalkyl;
R₉₈ is hydrogen, C₁-C₈alkyl, C₃-C₈alkenyl, C₃-C₈alkynyl, C₁-C₄haloalkyl or C₃-C₆haloalkenyl;
R₉₉ is hydrogen or C₁-C₈alkyl;
R₁₀₀ is hydrogen or C₁-C₈alkyl, or C₁-C₈alkyl substituted by one or more —COOH, C₁-C₈-alkoxycarbonyl or —CN substituents; or
R₁₀₀ is C₃-C₈alkenyl, C₃-C₈alkynyl, phenyl or benzyl, it being possible for phenyl and benzyl themselves to be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or
R₉₉ and R₁₀₀ together denote C₂-C₅alkylene; and
R₁₀₁ is hydrogen or C₁-C₈alkyl,
and an agrochemically acceptable salt or a stereoisomer or tautomer of a compound of formula I.

2. A process for the preparation of a compound of formula I according to claim 1, wherein a compound of formula II 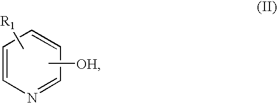
wherein R₁ is as defined for formula I, is reacted in the presence of a base with a compound of formula III 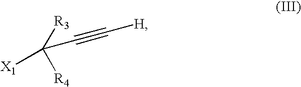
wherein R₃ and R₄ are as defined for formula I and X₁ is O-tosyl, chlorine, bromine or iodine, to form a compound of formula IV 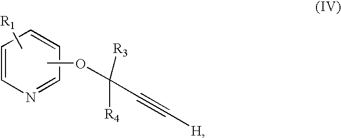
wherein R₁, R₃ and R₄ are as defined for formula 1, and that compound is then coupled, in the presence of a palladium catalyst, with a compound of formula V 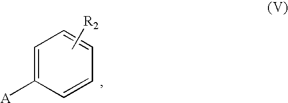
wherein R₂ is as defined for formula I and A is halogen or trifluoromethanesulfonate.

3. A herbicidal and plant-growth-inhibiting composition that comprises a herbicidally effective amount of a compound of formula I on an inert carrier.

4. A method of controlling undesired plant growth, which comprises applying a herbicidally effective amount of a compound of formula I or a composition comprising that compound to the plants or to the locus thereof.

5. A method of inhibiting plant growth, which comprises applying a herbicidally effective amount of a compound of formula I or a composition comprising that compound to the plants or to the locus thereof.

6. A compound according to claim 1, wherein each R₁ independently of any other(s) is halogen, —CN, —NO₂, 13 C(R₁₀)═NOR₁₁, —OR₁₃, —SO₂R₁₆, —OSO₂R₁₇, C₁-C₈alkyl or C₂-C₈alkenyl or C₁-C₈alkyl substituted by one or more halogen or —CN substituents;
R₁₀ is hydrogen or C₁-C₄alkyl; and
R₁₁ is C₁-C₈alkyl.

7. A compound according to claim 1, wherein
each R₂ independently of any other(s) is halogen, —CN, —NO₂, —NR₃₆R₃₇, —CO₂R₃₈, —C(R₄₁)═NOR₄₂, —OR₄₄, —SO₂R₄₇, —OSO₂R₄₈, C₁-C₈alkyl, or C₁-C₈alkyl mono- or poly-substituted by halogen, —CN or by —CO₂R₅₇;
R₃₈ and R₃₇ are hydrogen;
R₃₈ is hydrogen or C₁-C₈alkyl;
R₄₁ is hydrogen or C₁-C₄alkyl; and
R₄₂ is C₁-C₈alkyl.

8. A compound according to claim 1, wherein
each R₁ independently of any other(s) is halogen, —CN, —NO₂, —C(R₁₀)═NOR₁₁, —OR₁₃, —SO₂R₁₆, —OSO₂R₁₇, C₁-C₈alkyl or C₂-C₈alkenyl, or C₁-C₈alkyl substituted by one or more —CN;
R₁₀ is hydrogen or C₁-C₄alkyl;
R₁₁ is C₁-C₈alkyl;
each R₂ independently of any other(s) is halogen, —CN, —NO₂, —NR₃₆R₃₇, —CO₂R₃₈, —C(R₄₁)═NOR₄₂, —OR₄₄, —SO₂R₄₇, —OSO₂R₄₈ or C₁-C₈alkyl, or C₁-C₈alkyl mono- or poly-substituted by —CN or by —CO₂R₅₇;
R₃₆ and R₃₇ are hydrogen;
R₃₈ is hydrogen or C₁-C₈alkyl;
R₄₁ is hydrogen or C₁-C₄alkyl;
R₄₂ is C₁-C₈alkyl; and
R₃ and R₄ are each independently of the other hydrogen or C₁-C₄alkyl.

9. A compound according to claim 1, wherein
R₁ is halogen, —CN, C₁-C₈alkyl substituted by —CN, or C₁-C₈alkoxy.

10. A compound according to claim 1, wherein
R₂ is halogen, —CN, C₁-C₈alkyl substituted by —N, or C₁-C₈alkoxy.
